(19) United States
(12) Patent Application Publication
(10) Pub. No.: US 2002/0071842 A1
(43) Pub. Date: Jun. 13, 2002

(54) Soluble CD1 compositions and uses thereof

(76) Inventors: Jenny E. Gumperz, Jamaica Plain, MA (US); Michael B. Brenner, Newton, MA (US); Samuel M. Behar, Needham, MA (US)

Correspondence Address: Elizabeth R. Plumer, c/o Wolf, Greenfield & Sacks, P.C., Federal Reserve Plaza, 600 Atlantic Avenue, Boston, MA, 02210-2211, US

(21) Appl. No.: 09/874,470

(22) Filed: Jun. 5, 2001

Related U.S. Application Data
(60) Provisional application No. 60/209,416, filed on Jun. 5, 2000.

(51) Int. Cl. A61K 39/395; G01N 33/567

(52) U.S. Cl. 424/143.1; 435/7.21

(57) ABSTRACT

Compositions and methods for identifying CD1 antigens and CD1-restricted T cells, and diagnostic and therapeutic uses of same are provided. The compositions include CD1 fusion proteins, preferably multivalent fusion proteins that are present in multimeric form (e.g., by Protein A binding multiple CD1 fusion proteins).

### RELATED APPLICATIONS

This application claims domestic priority under 35 U.S.C. § 119(e) to U.S. Provisional Patent Application Ser. No. 60/209,416 filed Jun. 5, 2000, incorporated herein in its entirety by reference.

### GOVERNMENT SUPPORT

This invention was made in part with government support under grant numbers A128973 and CA47724 from the National Institutes of Health. The government may have certain rights in this invention.

### FIELD OF THE INVENTION

This invention relates to compositions and methods for identifying CD1-antigens and CD1-restricted T cells. The compositions include soluble CD1 molecules, particularly multimeric forms of soluble, divalent CD1 molecules. The compositions are useful for identifying CD1-restricted T cells in physiological samples and for modulating cellular immunity.

### BACKGROUND OF THE INVENTION

CD1 molecules are evolutionarily conserved P₂-microglobulin (β₂m) associated proteins, with a similar domain organization to class I antigen presenting molecules of the major histocompatibility complex (Porcelli, S. A., Adv. Immunol., 59:1-98 (1995)). However, CD1 molecules have a deeper and more hydrophobic antigen binding groove than class I molecules (Zeng et al., Science, 277:339-45 (1997)). Correspondingly, while class I molecules present peptide antigens, CD1 molecules can present lipids and glycolipids. Studies of human CD1a, b, and c molecules first demonstrated they can present microbial glycolipid antigens to T cells (Beckman, E. M. et al., J. Immunol., 157:2795-803 (1996); Beckman, E. M. et al., Nature, 372:691-4 (1994); Sieling, P. A. et al., Science, 269:227-30 (1995)). Subsequently, both human and murine CD1d molecules have been reported to present α-galactosylceramide (α-GalCer), a synthetic acylphytosphingolipid originally isolated from a marine sponge (Kawano, T. et al., Science, 278:1626-9 (1997); Spada, F. M. et al, J. Exp. Med., 188:1529-34 (1998)). However, the origin and the identity of the natural antigens recognized by CD1d-restricted T cells remain unknown. Accordingly, a need still existing to develop novel compositions and methods for identifying CD1 antigens and for identifying CD1 restricted T cells that are capable of presenting such naturally occurring antigens.

### SUMMARY OF THE INVENTION

The invention is based, in part, on the preparation of a stably folded, soluble form of CD1 and multimeric forms thereof, and on the discovery that such forms are useful for identifying CD1-specific antigens, and CD1-restricted T cells. In a preferred embodiment, the invention is based on the preparation of a stably folded soluble CD1 fusion protein that is multivalent and, optionally, fluorescently labeled, and that can be loaded with lipid or glycolipid antigens in vitro and used to stain or functionally investigate cognate T cells. Such fusion proteins of human CD1d and murine CD1d have been prepared and tested (see Examples), and are illustrative of the procedures that can be used to prepare and test the human CD1a, CD1b, CD1c, and CD1e fusion proteins, as well as to prepare and test the CD1 fusion proteins of other species, e.g., guinea pig, rabbit, rat, mouse, pig. Accordingly, the invention embraces compositions comprising soluble forms of any CD1 molecule and methods of using same as described herein.

According to one aspect of the invention, a method for identifying an antigen recognized by a CD1-restricted T cell is provided. The method involves:

(a) contacting a CD1 fusion protein with a putative CD1 antigen under conditions to form a CD1-presented antigen complex;

(b) contacting the CD1-presented antigen complex with a CD1-restricted T cell under conditions to allow complex-mediated activation of the T cell; and

(c) detecting activation of the T cell, wherein activation indicates that the putative CD1 antigen is recognized by the CD1 restricted T cell.

In certain embodiments, at least one contacting step (a) or (b) is performed in vitro; In these and other embodiments, at least one contacting step (a) or (b) is performed in vivo.

The preparation and characterization of an exemplary CD1 fusion protein, namely, CD1d-IgG fusion protein is described in the Examples. As used herein, a CD1 fusion protein refers to a soluble form of a CD1 molecule which retains a CD1 functional activity, i.e., the ability to selectively bind to a CD1 antigen to form a CD1-presented antigen complex (also referred to as a CD1-antigen complex); however, it is to be understood that other types of CD1 molecules (e.g., CD1a, CD1b, CD1c, CD1e), as well as other forms of a CD1 fusion protein (e.g., in which the IgG component is substituted by an alternative amino acid sequence, provided that the fusion protein is soluble and contains a CD1 molecule having a CD1 functional activity) are embraced by the instant invention.

In the preferred embodiments, the CD1 fusion protein is multimeric, i.e., the fusion protein contains two or more binding sites for the CD1 antigen. An exemplary, but non-limiting, method for preparing and characterizing a multimeric form of a CD1 fusion protein employs protein A to further form further multimeric structures, is provided in the Examples. Optionally, the protein A (or other agent which selectively binds to the CD1 molecule to form further multimers of the CD1 fusion protein) contains a detectable label for facilitating detection of the CD1 fusion protein in either isolated or bound form, e.g., bound to a CD1-restricted T cell, immobilized on a solid support.

CD1 molecules and certain characteristics of antigens that are presented by CD1 molecules previously have been described. (See, e.g., U.S. Pat. Nos. 5,679,347 and 5,853,737 and WO 95/00163; WO 96/12190; WO 99/12562; and WO 99/52547). Although non-mammalian CD1 antigens including, for example, mycobacterial antigens, have been described, CD1 antigens that are mammalian antigens (e.g., autoantigens) and plant antigens (e.g., allergens) have not been reported. Accordingly, the compositions and methods of the invention provide a means for identifying naturally-occurring antigens, as well as synthetic antigens (e.g., derived from a chemical library) that are selectively recognized and presented by CD1 molecules. In the preferred embodiments, the methods involve identifying novel antigens that are contained in or derived from a mammalian cell.

As used herein, a CD1-restricted T cell refers to a T cell that selectively recognizes a CD1-presented antigen. Exemplary CD1 restricted T cells are described in the Examples and include mouse NKT cells, mouse diverse CD1-restricted T cells (see, e.g., the Examples), as well as the following human T cell clones described in the literature: DN1.10B3; DN2.B9; DN2.D5; and DN2.D6.

As used herein, activation of a CD1-restricted T cell refers to a change in the T cell binding state or functional activity. Accordingly, detecting activation of the CD1-restricted T cell is accomplished by detecting one or more of the following parameters: (a) binding of the CD1-restricted T cell to a CD1-antigen complex; (b) a change in cytokine release by the CD1-restricted T cell; (c) a change in calcium flux in the CD1-restricted T cell; (d) a change in protein tyrosine phosphorylation level in the CD1-restricted T cell (e) phosphatidyl inositol turnover in the CD1-restricted T cell. Other detectable parameters that can be measured as indicators of the activation of a CD1-restricted T cell activity will be apparent to those of ordinary skill in the art. According to certain embodiments, particularly those involving human CD1-restricted T cells, the method preferably involves the further step of contacting the T cell with a co-stimulatory agent prior to detecting activation of the T cell (e.g., by contacting the T cells with anti-CD3 or other stimulant or co-stimulant).

Accordingly, the invention provides alternative types of screening methods for identifying putative CD1 antigens and putative CD1-restricted T cells. The first type of screening assay for identifying such antigens and cells involves two steps: (1) determining whether a putative CD1 antigen (“putative” or “test” compound) binds to a CD1 molecule (or conversely, whether a putative CD1-restricted T cell binds to a known CD1-presented CD1 antigen complex); and (2) determining whether the test compound selected in step (1) activates a CD1-restricted T cell. The second type of screening assay includes step (2) only, i.e., determining whether a putative CD1 antigen modulates a CD1-restricted T cell. Exemplary assays that are useful for practicing the two-step or one-step screening assay are discussed in more detail elsewhere in this application.

In general, the screening assays for detecting CD1 antigens and/or CD1 restricted T cells are tailored to measure a particular type of function, based on the nature of the putative compound. Thus, for example, CD1 antigens and CD1-restricted T cells that modulate a cellular immune response can be identified in screening assays which measure cytokine release or T cell proliferation. However, changes in cytokine profile also can be measured. For example, test compounds which shift the cytokine release profile to favor Th1 production or, conversely, to favor Th2 production, or which alter T cell proliferation to result in a change in immune response to an immunogen can be identified using the compositions and methods disclosed herein. Each of the foregoing types of screening assays are well known in the art; illustrative examples are provided below.

In certain embodiments, the putative CD1 antigens and/or putative CD1-restricted T cells can be identified by performing screening assays which detect the ability of a CD1-antigen complex (e.g., a fusion protein containing a putative CD1 antigen (“test compound”) or, conversely, a fusion protein containing a known CD1 antigen) to: (a) bind to a cognate CD1-restricted T cell (e.g., a putative CD1-restricted T cell or, conversely, a known CD1-restricted T cell) in a “binding assay”; (b) induce a change in a Th1/Th2 profile as indicated by an altered cytokine release profile (“cytokine release assay”) and/or antibody production (“antibody assay”) that is predictive of enhanced immunity; (c) induce a change in cell proliferation (“cell proliferation assay”) that is predictive of enhanced immunity; (d) enhance an immune response to infection (e.g., “infectious disease animal model”); (e) enhance vaccine-induced immunity (“vaccine animal model”); decrease an immune response to an autoimmune disorder or an allergic disorder (“autoimmune disease model”). Such screening assays are known in the art. Exemplary such assays are described in detail in the Examples and can be used to identify CD1 autoantigens and CD1-restricted T cells which satisfy the foregoing criteria.

Typically, the screening assays are performed in the presence and absence of a putative CD1 antigen or putative CD1-restricted antigen (“test compound”) and the effect of the test compound on the particular CD1-restricted T cell function being measured (e.g., binding to a CD1-presented antigen complex, cytokine release, cell proliferation, expression level) is determined. Putative CD1-antigens and CD1-restricted T cells that can be tested for the requisite functional activity include compounds that are present in libraries (e.g., libraries, such as small molecule medicinal pharmaceutical libraries), as well as compounds that are rationally designed to selectively bind to a CD1 molecule and, thereby, activate a cognate T cell. Thus, a compound is identified as a CD1 antigen if it: (1) binds to a CD1 molecule, and (2) modulates a CD1-restricted immune system response as determined using, for example, the assays provided herein and/or known to those of ordinary skill in the art.

Assays which measure cytokine release or cell proliferation are well known in the art. In general, the cytokine release assays of the invention detect the ability of a cell, preferably a CD1-restricted T cell, to release cytokine(s). Such assays may be performed in vivo or in vitro, with the in vitro cytokine release assays being predictive of an in vivo effect. Typically, cytokine release (e.g., release of one or more cytokines selected from the group consisting of: an interferon (e.g., IFN-gamma); an interleukin (e.g., IL-2, IL-4, IL-10, IL-13); a tumor necrosis factor (e.g., TNF-alpha); and a chemokine) is detected using immunoassays which selectively measure particular cytokines that are released by the cell. Exemplary cytokine release assays and their detection methods are provided in U.S. Ser. No. 60/115,055, filed Jan. 8, 1999, now abandoned; U.S. Ser. No. 09/473,937, filed Dec. 28, 1999, now pending; and PCT Application Ser. No. PCT US99/30992, filed Dec. 28, 1999 now published as WO 0040604, Jul. 13, 2000. Although not wishing to be bound to a particular theory or mechanism, it is believed that the CD1-antigen complexes of the invention alter the cytokine release profile of CD1-restricted T cells. In particular, the complexes of the invention may shift CD4+ CD1-restricted T cells towards a Th1 cytokine profile. Accordingly, the preferred cytokine release assays for use in accordance with the invention detect the ability of a putative CD1 antigen to increase the level of Th1 cytokines and/or decrease the level of Th2 cytokines released by a cell, preferably by a CD1-restricted T cell, relative to a cell which has not been contacted with the CD1-antigen complex.

According to yet another aspect of the invention, a method for identifying a CD1-restricted T cell is provided. The method involves:

(a) contacting a CD1-presented antigen complex with a putative CD1-restricted T cell under conditions to allow complex mediated activation of the putative CD1-restricted T cell; and

(b) detecting activation of the putative CD1-restricted T cell, wherein activation indicates that the putative CD1-restricted T cell is a CD1 restricted T cell. Complex-mediated activation of the CD1-restricted T cell is performed as disclosed with respect to the first aspect of the invention. In certain preferred embodiments, detecting activation of a putative CD1-restricted T cell involves detecting the CD1-presented complex containing a detectable label bound to the putative CD1-restricted T cell, e.g., by detecting the labeled T cells using flow cytometry. Sources of putative CD1-restricted T cells include biological samples, e.g., blood, cerebrospinal fluid, synovial fluid, tissue (e.g., biopsy), urine, amniotic fluid, peritoneal fluid, and gastric fluid.

In general, the screening assays of the invention involve: (1) determining a CD1-restricted T cell function in the absence of a complex comprising a CD1 fusion protein and a putative CD1 antigen (“test compound”), (2) determining a CD1-restricted T cell function in the presence of a complex comprising a CD1 fusion protein and a putative CD1 antigen; and (3) comparing the level of the CD1-restricted T cell function in the presence and absence of the test compound, wherein an increase in the level the CD1-restricted T cell function in the presence of the test compound indicates that the test compound is a CD1 antigen (“positive test compound”) that warrants further study to determine whether the positive test compound enhances an immune response. Thus, the preferred screening assays of the invention further include the step of performing an additional assay(s) to assess the ability of the positive test compounds to enhance an immune response. Such further assays include cell proliferation assays, infectious disease animal model assays, and vaccine animal model assays.

According to still another aspect of the invention, a method for detecting a CD1-restricted T cell activity in a sample is provided. The method is useful for diagnostic applications (see Examples) and involves the following steps:

(a) contacting a CD1-presented antigen complex with a sample suspected of containing a CD1-restricted T cell under conditions to allow complex mediated activation of the CD1-restricted T cell; and

(b) detecting a CD1-restricted T cell activity;

wherein the CD1-restricted T cell activity is selected from the group consisting of: (1) a CD1-restricted T cell concentration or a change in said concentration; and (2) a CD1-restricted T cell functional activity or a change in said functional activity.

In certain embodiments, detecting a CD1-restricted T cell activity involves detecting the concentration of the T cell (or a change in concentration of the T cell) in the sample (e.g., by flow cytometry). In yet other embodiments, detecting a CD1-restricted T cell activity involves detecting a CD1 restricted T cell functional activity (or a change in said functional activity). Exemplary CD1-restricted functional activities include: (a) binding of the CD1 restricted T cell to a CD1-antigen complex; (b) cytokine release by the CD1 restricted T cell; (c) calcium flux in the CD1 restricted T cell; (d) protein tyrosine phosphorylation in the CD1 restricted T cell; (e) phosphatidyl inositol turnover in the CD1 restricted T cell.

According to another aspect of the invention, a method for enhancing vaccine-induced acquired protective immunity is provided. The method involves administering to a subject a CD1 fusion protein in combination with a vaccine that enhances or induces protective immunity to a condition (e.g., an infectious disease, an allergic response, an autoimmune disorder, a cancer). In certain embodiments, the CD1 fusion protein is administered at the time of vaccination or, alternatively or additionally, subsequent to administering the vaccine to enhance recall of protective immunity. In general, the vaccine induces protective immunity to agents, particularly infectious agents such as microbes, allergens, autoantigens or tumor antigens, wherein Th1 cytokines are important for protective immunity to the condition. Exemplary infectious agents include agents which mediate a microbial infectious disease, such as tuberculosis, or which mediate a viral infectious disease, such as AIDS. Exemplary allergens, and tumor cell which can serve as sources of putative CD1 antigens are known in the art; illustrative examples are provided below.

According to a related aspect of the invention, a composition for practicing the foregoing method and methods for making same are provided. The composition generally includes: (1) an immunogen for inducing an immune response, (2) a CD1 fusion protein in an amount effective to enhance or induce protective immunity to a condition associated with the immunogen, and (3) a pharmaceutically acceptable carrier for vaccine use. Methods for making the composition involve placing the immunogen and the CD1 fusion protein in the pharmaceutically effective carrier. In one embodiment, the immunogen is an infectious agent (attenuated infectious agent or portion thereof) which may be selected or derived from the group consisting of bacteria, viruses, and parasites, and the amount of CD1 fusion protein contained in the composition is that amount effective to induce a protective immunity to a condition associated with an infectious agent (i.e., an infectious disease). In another embodiment, the immunogen is an allergen or an autoantigen and the CD1 fusion protein is provided in an amount effective to enhance or induce protective immunity to a condition associated with the allergen (e.g., an allergy) or autoimmune disorder. In yet another embodiment, the immunogen is a tumor antigen and the CD1 fusion protein is provided in an amount effective to enhance or induce protective immunity to a condition associated with the presence of the tumor antigen (i.e., a cancer).

In certain embodiments, the composition includes:

(a) a vaccine comprising an immunogen that: (1) selectively binds to a CD1 molecule, and (2) induces protective immunity to a disorder selected from the group consisting of: (a) an infectious disease; (b) a cancer; (c) an autoimmune disorder; and (d) an allergy,

(b) a CD1 fusion protein that selectively binds to the immunogen to form a CD1-immunogen complex that activates a cognate CD1-restricted T cell; wherein the CD1 fusion protein is present in an amount effective to enhance or induce protective immunity to the disorder, and a pharmaceutically acceptable carrier.

In the preferred embodiments, the CD1 fusion protein is multivalent and, more preferably, contains multiple CD1 fusion proteins (e.g., mediated by Protein A binding).

In general, a vaccine animal model is an animal model of acquired-immunity that is recognized by those of ordinary skill in the art as predictive of the ability of a vaccine to induce an acquired protective immunity to the infectious agent in humans. Such animal models detect the ability of a CD1 fusion protein-putative CD1 antigen complex to enhance a vaccine-induced acquired protective immunity and, thereby, are predictive of the efficacy of a putative CD1-restricted T cell CD1 antigen complex as an agent for enhancing protective immunity to the immunogen in humans. For example, such assays can detect a change in acquired resistance to a virulent infectious agent following inoculation of the animal with a non-virulent form of the infectious agent and administration of a putative CD1 antigen (alone or complexed with a CD1 fusion protein of the invention).

The foregoing assays are useful for identifying CD1 antigens for treating an infectious disease, cancer, and/or enhancing a vaccine-induced acquired protective immunity. Various aspects of the invention relating to these objectives are described below.

When the disorder is an infectious disease, the preferred immunogen is a lipid-containing molecule derived from an infectious agent selected from the group consisting of a bacterial infectious agent, a viral infectious agent, a fungal infectious agent, and a protist infectious agent. When the disorder is a cancer, the preferred immunogen is a lipid-containing molecule derived from a cancer cell. When the disorder is an allergy, the preferred immunogen is a lipid-containing molecule derived from allergens known to those of ordinary skill in the art. When the disorder is an autoimmune disorder, the immunogen is a lipid-containing molecule derived from a suspected autoimmune autoantigen.

In general, an infectious disease animal model is an animal model of a disease state that is recognized by those of ordinary skill in the art as a reasonable facsimile of the disease state in humans. Such animal models detect the ability of a putative CD1 antigen to ameliorate the symptoms of an infectious disease (e.g., M. tuberculosis) and, thereby, are predictive of the efficacy of the putative CD1 antigen complexed with the CD1 fusion proteins of the invention as a therapeutic agent for treating the infectious disease in humans. Typically, such assays detect a change in degree of infection (e.g., symptoms, infectious agent load, cytokine profile) following administration of a complex comprising a CD1 fusion protein-putative CD1 antigen to the animal. The compositions of the invention can be administered to the subject prior to the onset of the disorder (e.g., at time of vaccination) or during the disorder (e.g., infection, cancer diagnosis).

According to one aspect of the invention, a method of activation of antigen specific CD1-restricted T cells for immunotherapeutic treatment of disease (autoimmune disease, cancer, allergy, viral infections, bacterial infections) is provided. The method involves: (1) selecting antigen specific CD1-restricted T cells, e.g., by staining with the CD1-restricted T cell antigen complexes of the invention (optionally costimulating with a stimulatory agent), and (2) sterilely sorting the selected CD1-restricted T cells flow cytometry. The sorted T cells preferably are expanded in culture, e.g., by culturing in standard tissue culture medium containing phytohemagglutinin (PHA), IL-2, and irradiated autologous or allogeneic purified peripheral blood mononuclear “feeder” cells. This method causes the sorted T cells to proliferate in culture and therefore results in the expansion (and activation) of antigen-specific CD1-restricted T cells that can then be administered to patients for immunotherapy.

According to yet another embodiment of the invention, a method for depleting antigen specific CD1-restricted T cells for immunotherapeutic treatment of disease (autoimmune disease, cancer, allergy, viral infections, bacterial infections) is provided. The method involves: (1) selecting antigen specific CD1-restricted T cells, e.g., by staining with the CD1-restricted T cell antigen complexes of the invention (optionally costimulating with a stimulatory agent), and (2) sterilely sorting out (removing) the selected CD1-restricted T cells flow cytometry and (optionally) returning to the patient T cells which are not antigen specific CD1-restricted T cells. Thus, in this application the cells stained by the CD1 lipid antigen treated CD1 fusion protein aggregate are sorted out from the rest of the T cells and discarded, and the remaining T cells are readministered to the patient. Alternatively, a toxin is attached to the CD1 fusion protein and the antigen treated fusion protein aggregate is administered in vivo, to kill antigen specific CD1-restricted T cells.

These and other aspects of the invention, as well as various advantages and utilities, will be more apparent with reference to the detailed description of the preferred embodiments and to the accompanying drawings. Although the disclosure contains certain drawings, the drawings are not essential to the enablement of the claimed invention.

Certain terms used in this disclosure represent terms of art which have a meaning understood by one of ordinary skill in the art. Terms such as “effective amount” are defined in patents, such as those cited herein. Phrases such as “infectious disease”, “allergy”, “autoimmune disorder”, and “cancer” or “tumor antigen” have well-established meanings to those of ordinary skill in the art and are defined in standard medical texts. Examples of particular ranges of effective amounts and infectious diseases are provided herein for illustrative purposes only and are not intended to limit the scope of the invention. Thus, it will be understood that various modifications may be made to the embodiments disclosed herein without departing from the essence of the invention. Therefore, the description of the invention should not be construed as limiting, but merely as exemplifications of preferred embodiments. Those skilled in the art will envision other modifications within the scope of the claims appended hereto.

All documents and publications, including priority applications, if applicable, identified herein are incorporated in their entirely herein by reference.

### BRIEF DESCRIPTION OF THE DRAWINGS

The Examples may refer to and include a brief description of various figures and may refer to color representations. Certain of the referenced figures or color representations may not be present in this application as filed; however, it is to be understood that the drawings or colors which are not present are not essential to enablement of the inventions disclosed herein.

### DETAILED DESCRIPTION OF THE INVENTION

The invention is based, in part, on the preparation of a stably folded, soluble form of CD1 and multimeric forms thereof, and on the discovery that such forms are useful for identifying CD1-specific antigens, and CD1-restricted T cells. In a preferred embodiment, the invention is based on the preparation of a stably folded soluble CD1 fusion protein that is multivalent and, optionally, fluorescently labeled, and that can be loaded with lipid or glycolipid antigens in vitro and used to selectively stain or functionally investigate cognate T cells. Such fusion proteins of human CD1d and murine CD1d have been prepared and tested (see the Examples), and are illustrative of the procedures that can be used to prepare and test the human CD1a, CD1b, CD1c, and CD1e molecules. Accordingly, the invention embraces compositions comprising soluble forms of any CD1 molecule and methods of using same as described herein.

Screening Methods and Compositions of Matter:

The compositions and methods disclosed herein are useful for identifying agents which are useful for treating immune related disease such as infectious diseases, allergies, autoimmunity, and cancer, for diagnostic applications, and/or for enhancing vaccine-induced acquired protective immunity for the purpose of treating these conditions.

(1) Screening Methods to Identify Putative CD1 Antigens:

According to one aspect of the invention, a method for identifying an antigen recognized by a CD1-restricted T cell is provided. The method involves:

(a) contacting a CD1 fusion protein with a putative CD1 antigen under conditions to form a CD1-presented antigen complex;

(b) contacting the CD1-presented antigen complex with a CD1-restricted T cell under conditions to allow complex-mediated activation of the T cell; and

(c) detecting activation of the T cell, wherein activation indicates that the putative CD1 antigen is recognized by the CD1 restricted T cell.

In certain embodiments, at least one contacting step (a) or (b) is performed in vitro; In yet other embodiments, at least one contacting step (a) or (b) is performed in vivo.

The preparation and characterization of an exemplary CD1 fusion protein, namely, CD1d-IgG fusion protein is described in the Examples. As used herein, a CD1 fusion protein refers to a soluble form of a CD1 molecule which retains a CD1 functional activity, i.e., the ability to selectively bind to a CD1 antigen to form a CD1-antigen complex; however, it is to be understood that other types of CD1 molecules (e.g., CD1a, CD1b, CD1c, CD1e), as well as other forms of a CD1 fusion protein (e.g., in which the IgG component is substituted by an alternative amino acid sequence, provided that the fusion protein is soluble and contains a CD1 molecule having a CD1 functional activity) are embraced by the instant invention.

In the preferred embodiments, the CD1 fusion protein is multimeric, i.e., the fusion protein contains two or more binding sites for the CD1 antigen. An exemplary, but non-limiting, method for preparing and characterizing a multimeric form of a CD1 fusion protein that employs Protein A to form further multimers of the CD1 fusion protein structure is provided in the Examples. The multimers retain the functional activity of the CD1 fustion protein. Optionally, the Protein A (or other agent which selectively binds to the CD1 molecule) contains a detectable label for facilitating detection of the CD1 fusion protein in either isolated or bound form, e.g., bound to a CD1-restricted T cell, immobilized on a solid support. Other methods for forming further multimeric forms of soluble CD1 fusion molecules that retain their ability to present CD1 antigens are based on methods reported in the art for forming multimers of other types of ligand-binding proteins. For example, amino acid sequences which can be biotinylated can be incorporated into a CD1 fusion protein, thereby allowing for avidin-induced multimerization of the CD1 fusion protein. (See, e.g., Altman, J. D., et al., Science, 274:94-6 (1996); Crawford, F., et al., Immunity, 8:675-82 (1998); Gutgemann, T., et al., Immunity, 8:667-73 (1998); Busch, D., Immunity, 8:353-62 (1998); Kerksiek, K. M., et al., J. Exp. Med., 190(2):195-204 (1999); and Crowley, M. P., Science 287(5451):413-6 (2000).

CD1 molecules and selected characteristics of mycobacterial antigens that are presented by CD1 molecules previously have been described. (See, e.g., U.S. Pat. Nos. 5,679,347 and 5,853,737 and WO 95/00163; WO 96/12190; WO 99/12562; and WO 99/52547). In general, the CD1 antigens of the invention are naturally-occurring, lipid-containing molecules or synthetic molecules with at least some hydrophobic component(s) that mimic the lipid-like properties of a naturally occuring CD1 antigen. Preferably, the putative CD1 antigen is a lipid containing molecule selected from the group consisting of: a polar lipid (e.g., a ganglioside, a phospholipid); a neutral lipid, a glycolipid; and a lipidated protein or lipidated peptide. In certain embodiments, the putative CD1 antigen is contained in or isolated from a sample selected from the group consisting of: a mammalian cell, a plant cell, a bacteria, a virus, a fungus, a protist, and a synthetic library. In other embodiments, the putative CD1 antigen is contained in or isolated from a total lipid extract of a sample selected from the group consisting of: a mammalian cell, a plant cell, a bacteria, a virus, a fungus, a protist, and a synthetic library. Although non-mammalian CD1 antigens including, for example, mycobacterial antigens, have been described, CD1 antigens that are mammalian antigens (e.g., autoantigens) and plant antigens (e.g., allergens) have not been reported. Accordingly, the compositions and methods of the invention provide a means for identifying naturally-occurring antigens, as well as synthetic antigens (e.g., derived from a chemical library) that are selectively recognized and presented by CD1 molecules. In the preferred embodiments, the methods involve identifying novel lipid-containing antigens that are contained in or derived from a mammalian cell, e.g., by whole lipid extraction. In preferred embodiments, the putative CD1 antigen is a mammalian cell that is contained in or derived from a sample selected from the group consisting of: a blood sample, a cerebrospinal fluid sample, a synovial fluid sample, a tissue sample, a urine sample, an amniotic fluid sample, a peritoneal fluid sample, and a gastric fluid sample.

In a general sense, the invention embraces screening various types of libraries to identify putative CD1 antigens, including naturally-occurring and synthetic antigens. Putative CD1 antigens can be synthesized using recombinant or chemical library approaches. A vast array of putative CD1 antigens can be generated from libraries of synthetic or natural compounds. Libraries of natural compounds in the form of bacterial, fungal, plant and animal extracts are available or can readily produced. Whole lipid extracts of the foregoing natural sources are preferred sources of putative CD1 antigens for testing in accordance with the methods of the invention. Natural and synthetically produced libraries and compounds can be readily modified through conventional chemical, physical, and biochemical means. Known CD1 antigens such as those derived from mycobacteria or any of the CD1-antigens mentioned herein, may be subjected to directed or random chemical modifications such as acylation, alkylation, esterification, amidification, etc. to produce structural analogs of these binding partners, which function as CD1 antigens.

The methods of the invention utilize library technology to identify small molecules including small glycolipids which bind to the CD1 fusion proteins of the invention. One advantage of using libraries for CD1 antigen identification is the facile manipulation of millions of different putative candidates of small size in small reaction volumes (i.e., in synthesis and screening reactions). Another advantage of libraries is the ability to synthesize CD1 antigens which might not otherwise be attainable using naturally occurring sources.

Methods for preparing libraries of molecules are well known in the art and many libraries are commercially available. Small molecule combinatorial libraries may be generated. A combinatorial library of small organic compounds is a collection of closely related analogs that differ from each other in one or more points of diversity and are synthesized by organic techniques using multi-step processes. Combinatorial libraries include a vast number of small organic compounds. One type of combinatorial library is prepared by means of parallel synthesis methods to produce a compound array. A “compound array” as used herein is a collection of compounds identifiable by their spatial addresses in Cartesian coordinates and arranged such that each compound has a common molecular core and one or more variable structural diversity elements. The compounds in such a compound array are produced in parallel in separate reaction vessels, with each compound identified and tracked by its spatial address. Examples of parallel synthesis mixtures and parallel synthesis methods are provided in U.S. Ser. No. 08/177,497, filed Jan. 5, 1994 and its corresponding PCT published patent application WO95/18972, published Jul. 13, 1995 and U.S. Pat. No. 5,712,171 granted Jan. 27, 1998 and its corresponding PCT published patent application WO96/22529, which are hereby incorporated by reference.

The putative CD1 antigens, isolated or contained in a mixture or library, are contacted with the CD1 fusion proteins of the invention to form CD1-presented antigen complexes which, in turn, are contacted with CD1-restricted T cells to determine whether the T cell selectively recognizes the putative antigen. Thus, as used herein, a CD1-restricted T cell refers to a T cell that selectively recognizes a CD1-presented antigen and, preferably, is activated by contact with the CD1-presented antigen complex to alter its functional activity. Exemplary CD1 restricted T cells are described in the Examples and include mouse NKT cells, as well as the following human T cell clones previously described in the literature: DN1.10B3; DN2.B9; DN2.D5; and DN2.D6.

As used herein, activation of a CD1-restricted T cell refers to a change in a binding state or functional activity of the CD1-restricted T cell. Accordingly, detecting activation of the CD1-restricted T cell is accomplished by detecting one or more of the following parameters: (a) binding of the CD1-restricted T cell to a CD1-presented antigen complex; (b) a change in cytokine release by the CD1-restricted T cell; (c) a change in calcium flux in the CD1-restricted T cell; (d) a change in protein tyrosine phosphorylation flux in the CD1-restricted T cell (e) phosphatidyl inositol turnover flux in the CD1-restricted T cell. Other detectable parameters that can be measured as indicators of activation of a CD1-restricted T cell activity will be apparent to those of ordinary skill in the art. According to certain embodiments, particularly those involving human CD1-restricted T cells, the method for detecting T cell activation preferably further includes the step of contacting the T cell with a co-stimulatory agent prior to detecting activation of the T cell (e.g., by contacting the cells with anti-TCR, anti-CD3 or other stimulant). Exemplary co-stimulatory agents include agents selected from the group consisting of: (a) an adhesion molecule (e.g., CD2); (b) an NK complex molecule (e.g., CD161, CD94); (c) an antibody to the T cell receptor (e.g., an anti-CD3 antibody); (d) a non-specific stimulator (e.g., phytohemaglutinin (“PHA”), concanavalin A (Con A”); phorbol myristate acetate (“PMA”); (e) an antigen-presenting cell which does not express CD1; and (f) a co-stimulatory molecule (e.g., CD28).

In general, the screening assays for detecting CD1 antigens and/or CD1 restricted T cells are tailored to measure a particular type of CD1-restricted T cell function, based on the nature of the putative CD1 antigen. For example, CD1 antigens and CD1-restricted T cells (that modulate a cellular immune response) can be identified in screening assays which measure cytokine release or T cell proliferation. Thus, for example, test compounds which induce cytokine release or which shift the cytokine release profile to favor Th1 production or, conversely, to favor Th2 production, or which alter T cell proliferation, thereby resulting in a change in immune response to an immunogen, can be identified using the compositions and methods disclosed herein.

In summary, the invention provides alternative types of screening methods for identifying putative CD1 antigens and putative CD1-restricted T cells. The first type of screening assay for identifying such antigens and cells involves two steps: (1) determining whether a putative CD1 antigen (“putative” or “test” compound) binds to a CD1 molecule (or conversely, whether a putative CD1-restricted T cell recognizes (e.g., binds to a known CD1-presented antigen); and (2) determining whether the test compound selected in step (1) activates a CD1-restricted T cell. The second type of screening assay includes step (2) only, i.e., determining whether a putative CD1 antigen activates a CD1-restricted T cell.

In certain embodiments, the putative CD1 antigens and/or putative CD1-restricted T cells can be identified by performing screening assays which detect the ability of a CD1-presented antigen complex (e.g., a CD1 fusion protein associated with a putative CD1 antigen (“test compound”) or, conversely, a fusion protein containing a known CD1 antigen) to: (a) bind to a cognate CD1-restricted T cell (e.g., a known CD1-restricted T cell) or conversely, a putative CD1-restricted T cell) in a “binding assay”; (b) induce a change in a Th1/Th2 profile as indicated by an altered cytokine release profile (“cytokine release assay”) and/or antibody production (“antibody assay”) that is predictive of enhanced immunity; (c) induce a change in cell proliferation (“cell proliferation assay”) that is predictive of enhanced immunity; (d) enhance an immune response to infection (e.g., “infectious disease animal model”); (e) enhance vaccine-induced immunity (“vaccine animal model”); (f) decrease an immune response to an autoimmune disorder (“autoimmune disease model”); or (g) decrease an allergic disorder (“allergic disease model”). Such screening assays are known in the art and can be used in accordance with the methods and compositions of the invention to identify CD1 autoantigens and CD1-restricted T cells which satisfy the foregoing binding and activation criteria.

Typically, the screening assays are performed in the presence and absence of a putative CD1 antigen or putative CD1-restricted antigen (“test compound”) and the effect of the test compound on the particular CD1-restricted T cell function being measured (e.g., binding to a CD1-presented antigen complex, cytokine release, cell proliferation, expression level) is determined. Putative CD1-antigens and CD1-restricted T cells that can be tested for the requisite functional activity include compounds that are present in libraries (e.g., libraries, such as small molecule medicinal pharmaceutical libraries), as well as compounds that are rationally designed to selectively bind to a CD1 molecule and, thereby, activate a cognate T cell.

A compound is identified as a CD1 antigen if it: (1) binds to a CD1 molecule, and (2) modulates a CD1-restricted immune system response as determined using, for example, the assays provided herein and/or those known to those of ordinary skill in the art. For example, assays which measure cytokine release or cell proliferation are well known in the art. In general, the cytokine release assays of the invention detect the ability of a CD1-restricted T cell to release cytokine(s). Such assays may be performed in vivo or in vitro, with the in vitro cytokine release assays being predictive of an in vivo effect. Typically, cytokine release is detected using immunoassays which selectively measure particular cytokines that are released by the cell. Exemplary cytokine release assays and their detection methods are provided in U.S. Ser. No. 60/115,055, filed Jan. 8, 1999, now abandoned; U.S. Ser. No. 09/473,937, filed Dec. 28, 1999, now pending; and PCT Application Serial No. PCT US99/30992, filed Dec. 28, 1999 and published as WO 0040604, Jul. 13, 2000. Although not wishing to be bound to a particular theory or mechanism, it is believed that the CD1-antigen complexes of the invention alter the cytokine release profile of CD1-restricted T cells. In particular, the complexes of the invention may shift CD4+ CD1-restricted T cells towards a Th1 cytokine profile. Accordingly, the preferred cytokine release assays for use in accordance with the invention detect the ability of a putative CD1 antigen to increase the level of Th1 cytokines and/or decrease the level of Th2 cytokines released by a cell, preferably by a CD1-restricted T cell, relative to a CD-restricted T cell which has not been contacted with the CD1-fusion protein presented antigen complex.

(2) Screening Methods to Identify Putative CD1-restricted T cells:

According to yet another aspect of the invention, a method for identifying a CD1-restricted T cell is provided. The method involves:

(a) contacting a CD1-presented antigen complex with a putative CD1-restricted T cell under conditions to allow complex mediated activation of the putative CD1-restricted T cell; and

(b) detecting activation of the putative CD1-restricted T cell, wherein activation indicates that the putative CD1-restricted T cell is a CD1 restricted T cell. Complex-mediated activation of the CD1-restricted T cell is performed as disclosed with respect to the first aspect of the invention.

In certain preferred embodiments, detecting activation of a putative CD1-restricted T cell involves detecting the CD1-presented complex containing a detectable label bound to the putative CD1-restricted T cell, e.g., by detecting the labeled T cells using flow cytometry. Sources of putative CD1-restricted T cells include biological samples, e.g., blood, cerebrospinal fluid, synovial fluid, tissue (e.g., biopsy), urine, amniotic fluid, peritoneal fluid, and gastric fluid.

Diagnostic Methods:

According to still another aspect of the invention, a method for detecting a CD1-restricted T cell activity in (or isolated from) a sample, e.g., a peripheral blood sample is provided. (See, e.g., the Examples.) The method involves:

(a) contacting a CD1-presented antigen complex with a sample suspected of containing a CD1-restricted T cell under conditions to allow complex mediated activation of the CD1-restricted T cell; and

(b) detecting a CD1-restricted T cell activity;

wherein the CD1-restricted T cell activity is selected from the group consisting of: (1) the number of CD1-restricted T cells as a percentage of the total T cell population or a change in said number; and (2) a CD1-restricted T cell functional activity or a change in said functional activity.

In certain embodiments, detecting a CD1-restricted T cell activity involves detecting the number of the CD1-restricted T cells (or a change in the number of the CD1-restricted T cells) in the sample (e.g., by flow cytometry). In yet other embodiments, detecting a CD1-restricted T cell activity involves detecting a CD1 restricted T cell functional activity (or a change in said functional activity). Exemplary CD1-restricted functional activities include: (a) binding of the CD1 restricted T cell to a CD1-antigen complex; (b) cytokine release by the CD1 restricted T cell; (c) calcium flux in the CD1 restricted T cell; (d) protein tyrosine phosphorylation in the CD1 restricted T cell; (e) phosphatidyl inositol turnover in the CD1 restricted T cell.

Samples that can be tested for the presence/activity of a CD1-restricted antigen include samples selected from the group consisting of a blood sample, a cerebrospinal fluid sample, a synovial fluid sample, a tissue sample, a urine sample, an amniotic fluid sample, a peritoneal fluid sample, and a gastric fluid sample. An illustrative example of a diagnostic method is provided in the Examples.

Therapeutic Methods and Compositions:

As noted throughout this application, the CD1 antigens that are useful for treating various disorders can be identified (e.g., isolated from naturally occurring infectious agents, tumor antigens, allergens, and autoantigens) using the screening methods disclosed herein. The following paragraphs provide examples of immunogens for the representative disorders. These immunogens can be used as a source of lipid-containing putative CD1 antigens for identification in the screening assays.

To be useful in the therapeutic methods described herein, the CD1 antigens (either presently known or identified, e.g., using the screening methods of the invention) when presented by the CD1 fusion proteins of the invention must also be capable of modulating an immune response. In certain instances, such modulation is accompanied by cytokine release by a CD1-restricted T cell or a shift in cytokine release profile by a CD1-restricted T cell. For example, such CD1-presented antigen complexes may enhance a Th1 response or a Th2 response. Thus, in some embodiments such as those aimed at preventing allergic reactions or reducing an autoimmune response, the complexes of the invention are those which down-regulate a Th1 response or a Th2 response to achieve a therapeutic effect.

It should be noted that the invention intends to embrace any treatment regimen in which an increased Th1 or Th2 cytokine response or antibody response, or alternatively, when appropriate to achieve a therapeutic effect, a decreased Th1 or Th2 cytokine response against an immunogen would have a therapeutic benefit. As described above, such immunizations include infectious agents, allergens, autoantigens, and tumor antigens.

Vaccine-induced acquired protective immunity as used herein refers to an immunity which occurs as a result of deliberate exposure with an immunogen in a form and/or dose which does not induce an illness (such as an infectious disease) or a disorder (such as an allergic reaction) in a subject. The deliberate exposure generally takes the form of a vaccine which contains an immunogen which is administered to a subject in order to stimulate an immune response to the immunogen and, thereby, render the subject immune to subsequent challenge with the immunogen. The invention therefore provides methods and compositions for enhancing vaccine induced immunity by administering a vaccine, in any of the forms described herein, in the context of CD1 antigen presentation. Thus, the method involves administering to a subject a CD1 fusion protein in combination with a vaccine that induces protective immunity. “Administering in combination” embraces administration of a CD1 fusion protein prior to, concurrently with or following the administration of a vaccine. In some preferred embodiments, the CD1 fusion protein is administered substantially simultaneously with the vaccine, so that CD1 presentment of the immunogen occurs at the time of the initial immune response. For the purpose of mass vaccination, this latter method of incorporating a CD1 fusion protein in a vaccine composition is preferred. In still other embodiments, the CD1 fusion protein loaded with CD1 antigen is administered to the subject subsequent to (i.e., following) the administration of the vaccine in order to enhance recall of protective immunity. This latter method may be more appropriate, for example, in animal screening models. Protective immunity refers to an immunity that is developed after a primary infection and which the subject possesses for long periods of time (potentially even for a life-time) following the primary infection. As such, the subject's immune system is able to mount effectively a response to the antigen upon subsequent exposure, thereby preventing subsequent infection or disease. In preferred embodiments, the vaccine contains an infectious agent, or an immunogen, which will stimulate an immune response within the subject. The immunogen can be derived from infectious bacteria, an infectious virus, an infectious fungus or an infectious parasite such as a protist. Thus, the method for enhancing vaccine-induced acquired protective immunity can be directed towards the treatment of microbial infectious disease.

According to one aspect of the invention, a method for enhancing vaccine-induced acquired protective immunity is provided. The method involves administering to a subject a CD1 fusion protein in combination with a vaccine that enhances or induces protective immunity to a condition (e.g., an infectious disease, an allergic response, an antoimmune disorder, a cancer). In certain embodiments, the CD1 fusion protein is administered at the time of vaccination or, alternatively or additionally, subsequent to administering the vaccine to enhance recall of protective immunity. In general, the vaccine induces protective immunity to agents, particularly infectious agents such as microbes, allergens, or tumor antigens, wherein Th1 cytokines are important for protective immunity to the condition. Exemplary infectious agents include agents which mediate a microbial infectious disease, such as tuberculosis, or which mediate a viral infectious disease, such as AIDS. Exemplary allergens, and tumor antigens are known in the art; illustrative examples are described below.

(1) Treatment of Infectious Disease:

In one aspect, the invention provides a method for treating an infectious disease. The method involves administering an effective amount of a CD1 fusion protein of the invention, preferably in combination with a CD1 antigen to induce an immune response to the infectious disease, to a subject in need of such treatment. As used herein, the amount effective to treat the subject is that amount which inhibits either the development or the progression of a disorder or decreases the rate of progression of the disorder, e.g., an infectious disease.

The treatment methods described herein also embrace prophylactic treatment, e.g., of an infectious disease. The prophylactic method may further comprise, in another embodiment, the selection of a subject at risk of developing a disorder prior to the administration of the agent. Subjects at risk of developing an infectious disease include those who are likely to be exposed to an infectious agent. As example of such a subject is one who has been in contact with an infected subject, or one who is travelling or has traveled to a location in which a particular infectious disease in endemic. The prophylactic treatment methods provided may also include an initial step of identifying a subject at risk of developing an infectious disease. In some preferred embodiments, the prophylactic treatment may involve administering a vaccine to a subject.

As defined herein, an infectious disease or infectious disorder is a disease arising from the presence of a microbial agent in the body. The microbial agent may be an infectious bacteria, an infectious virus, an infectious fungi, or an infectious protist (such as a parasite).

Examples of infectious bacteria include but are not limited to: Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, Actinomyces israelli, and Salmonella spp.

Examples of infectious virus include but are not limited to: Retroviridae (e.g. human immunodeficiency viruses, such as HIV-1 (also referred to as HTLV-III, LAV or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviradae (e.g. vesicular stomatitis viruses, rabies viruses); Coronaviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxyiridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Examples of infectious fungi include: Cryptococcus neoformans, Histoplasma capsulatum, Coccidioides immitis, Blastomyces dermatitidis, Chlamydia trachomatis, Candida albicans. Other infectious organisms (i.e., protists) include: Plasmodium such as Plasmodium falciparum, Plasmodium knowlesi, Plasmodium malariae, Plasmodium ovale, Plasmodium malariae, and Plasmodium vivax, and Toxoplasma gondii, Babesia microti, Babesia divergens, Trypanosoma cruzi, Trichinella spiralis, Leishmania major, Leishmania donovani, Leishmania braziliensis Leishmania tropica, and Giardia spp.

In preferred embodiments, the microbial agent is one which causes a disease, the progression of which can be inhibited or halted by the presence of Th1 T cells and/or Th1cytokines. Infectious diseases which can favorably be treated with Th1 cytokines include those caused by microbial agents, examples of which are salmonellosis and tuberculosis.

(2). Treatment of Cancers:

Generally the tumor antigen of choice will be a lipid-containing molecule which binds to any of the CD1 molecules to form a complex which activates a CD1-restricted T-cell. Typically, such antigens can be isolated from whole lipid extracts of tissue or other samples containing the tumor cells of the particular cancer being treated. Such antigens are identified using the screening assays disclosed herein. Cancers to be treated using the methods and compositions of the invention are preferably those which would benefit from an enhanced Th1 response. Examples of these include but are not limited to biliary tract cancer; brain cancer, including glioblastomas and medulloblastomas; breast cancer; cervical cancer; choriocarcinoma; colon cancer; endometrial cancer; esophageal cancer; gastric cancer; hematological neoplasms, including acute lymphocytic and myelogenous leukemia; chronic lymphocytic and myelogenous leukemia, multiple myeloma; AIDS associated leukemias and adult T-cell leukemia lymphoma; intraepithelial neoplasms, including Bowen's disease and Paget's disease; liver cancer; lung cancer; lymphomas, including Hodgkin's disease and lymphocytic lymphomas; neuroblastomas; oral cancer, including squamous cell carcinoma; ovarian cancer, including those arising from epithelial cells, stromal cells, germ cells and mesenchymal cells; pancreas cancer; prostate cancer; colorectal cancer; sarcomas, including leiomyosarcoma, rhabdomyosarcoma, liposarcoma, fibrosarcoma and osteosarcoma; skin cancer, including melanoma, Kaposi's sarcoma, basocellular cancer and squamous cell cancer; testicular cancer, including germinal tumors (seminoma, non-seminoma teratomas and choriocarcinomas), stromal tumors and germ cell tumors; thyroid cancer, including thyroid adenocarcinoma and medullar carcinoma; and renal cancer including adenocarcinoma and Wilms' tumor.

(3). Treatment of Allergies:

An “allergy” as used herein refers to acquired hypersensitivity to a substance (i.e., an allergen). Allergic conditions or diseases in humans include but are not limited to eczema, allergic rhinitis or coryza, hay fever, conjunctivitis, bronchial or allergic asthma, urticaria (hives) and food allergies; atopic dermatitis; anaphylaxis; drug allergy; angioedema; and allergic conjunctivitis. Allergic diseases in dogs include but are not limited to seasonal dermatitis; perennial dermatitis; rhinitis: conjunctivitis; allergic asthma; and drug reactions. Allergic diseases in cats include but are not limited to dermatitis and respiratory disorders; and food allergens. Allergic diseases in horses include but are not limited to respiratory disorders such as “heaves” and dermatitis. Allergic diseases in non-human primates include but are not limited to allergic asthma and allergic dermatitis.

The generic name for molecules that cause an allergic reaction is allergen. There are numerous species of allergens. The allergic reaction occurs when tissue-sensitizing immunoglobulin of the IgE type reacts with foreign allergen. The IgE antibody is bound to mast cells and/or basophils, and these specialized cells release chemical mediators (vasoactive amines) of the allergic reaction when stimulated to do so by allergens bridging the ends of the antibody molecule. Histamine, platelet activating factor, arachidonic acid metabolites, and serotonin are among the best known mediators of allergic reactions in man. Histamine and the other vasoactive amines are normally stored in mast cells and basophil leucocytes. The mast cells are dispersed throughout animal tissue and the basophils circulate within the vascular system. These cells manufacture and store histamine within the cell unless the specialized sequence of events involving IgE binding occurs to trigger its release.

Allergens include but are not limited to Environmental Aeroallergens; plant pollens such as Ragweed/hayfever (affects 10% of pop., 25 million ppl); Weed pollen allergens; Grass pollen allergens (grasses affect 10% of pop., 25 million ppl); Johnson grass; Tree pollen allergens; Ryegrass; House dust mite allergens (affects 6% of pop., 15 million ppl); Storage mite allergens; Japanese cedar pollen/hay fever (affects 10% of pop. In Japan, 13 million ppl); Mold spore allergens; Animal allergens (cat (affects 2% of pop., 5 million ppl), dog, guinea pig, hamster, gerbil, rat, mouse); Food Allergens (e.g., Crustaceans; nuts, such as peanuts; citrus fruits); Insect Allergens (Other than mites listed above); Venoms: (Hymenoptera, yellow jacket, honey bee, wasp, hornet, fire ant); Other environmental insect allergens from cockroaches, fleas, mosquitoes, etc.; Bacteria such as streptococcal antigens; Parasites such as Ascaris antigen; Viral Antigens; Fungal spores; Drug Allergens; Antibiotics; penicillins and related compounds; other antibiotics; Whole Proteins such as hormones (insulin), enzymes (Streptokinase); all drugs and their metabolites capable of acting as incomplete antigens or haptens; Industrial Chemicals and metabolites capable of acting as haptens and stimulating the immune system (Examples are the acid anhydrides (such as trimellitic anhydride) and the isocyanates (such as toluene diisocyanate)); Occupational Allergens such as flour (ie. Baker's asthma), castor bean, coffee bean, and industrial chemicals described above; flea allergens; and human proteins in non-human animals.

Examples of specific natural, animal and plant allergens include but are not limited to lipids, including glycolipids and lipoproteins, specific to the following genuses: Canine (Canis familiaris); Dermatophagoides (e.g. Dermatophagoides farinae); Felis (Felis domesticus); Ambrosia (Ambrosia artemiisfolia; Lolium (e.g. Lolium perenne or Lolium multiflorum); Cryptomeria (Cryptomeria japonica); Alternaria (Alternaria alternata); Alder; Alnus (Alnus gultinoasa); Betula (Betula verrucosa); Quercus (Quercus alba); Olea (Olea europa); Artemisia (Artemisia vulgaris); Plantago (e.g. Plantago lanceolata); Parietaria (e.g. Parietaria officinalis or Parietaria judaica); Blattella (e.g. Blattella germanica); Apis (e.g. Apis multiflorum); Cupressus (e.g. Cupressus sempervirens, Cupressus arizonica and Cupressus macrocarpa); Juniperus (e.g. Juniperus sabinoides, Juniperus virginiana, Juniperus communis and Juniperus ashei); Thuya (e.g. Thuya orientalis); Chamaecyparis (e.g. Chamaecyparis obtusa); Periplaneta (e.g. Periplaneta americana); Agropyron (e.g. Agropyron repens); Secale (e.g. Secale cereale); Triticum (e.g. Triticum aestivum); Dactylis (e.g. Dactylis glomerata); Festuca (e.g. Festuca elatior); Poa (e.g. Poa pratensis or Poa compressa); Avena (e.g. Avena sativa); Holcus (e.g. Holcus lanatus); Anthoxanthum (e.g. Anthoxanthum odoratum); Arrhenatherum (e.g. Arrhenatherum elatius); Agrostis (e.g. Agrostis alba); Phleum (e.g. Phleum pratense); Phalaris (e.g. Phalaris arundinacea); Paspalum (e.g. Paspalum notatum); Sorghum (e.g. Sorghum halepensis); and Bromus (e.g. Bromus inermis).

In general, the pharmaceutical compositions of the invention include the CD1 fusion proteins (alone, loaded with CD1 antigens or otherwise in combination with an immunogen) in combination with any standard physiologically and/or pharmaceutically acceptable carriers which are known in the art. The compositions should be sterile and contain a therapeutically effective amount of the active ingredients in a unit of weight or volume suitable for administration to a patient. The term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art.

The invention further provides compositions useful in enhancing vaccine-induced acquired protective immunity. Such compositions include a vaccine comprising an immunogen (e.g., and infectious agent or an infectious fragment thereof), a CD1 fusion protein in an amount effective, for examples, in this instance, to enhance or induce protective immunity to the infectious agent or fragment thereof, and a pharmaceutically acceptable carrier. Exemplary conditions that are mediated by an abnormally reduced level of Th1 cytokines or which would benefit from an increased level of Th1 cytokines include infectious diseases (e.g., tuberculosis, Salmonella infection). In yet another embodiment, conditions that are mediated by an abnormally increased level of Th2 cytokines or which would benefit from a decreased level of Th2 cytokines could be treated using the compositions and methods described herein relating to a vaccine-induced acquired protective immunity. As example of these latter conditions include allergic responses, particularly in a subject who is susceptible to allergies. A highly allergic subject could be administered a vaccine which comprises an CD1 fusion protein and a suspect immunogen (i.e., an allergen). In this way, the subject is immunized to the suspect allergen in the absence of an adverse Th2 allergic response. Rather the subject experiences the allergen in the context of an CD1 fusion protein, and thus in the presence of a Th1 immune response. In compositions which include an allergen, the allergen is present in an amount effective to enhance or induce protective immunity to the allergen. As example of an effective amount is the amount required for the prevention of an allergic response to subsequent challenges with the allergen.

The pharmaceutical preparations, as described above, are administered in effective amounts. The effective amount will depend upon the mode of administration, the particular condition being treated and the desired outcome. It will also depend upon, as discussed above, the stage of the condition, the age and physical condition of the subject, the nature of concurrent therapy, if any, and like factors well known to the medical practitioner. For therapeutic applications, it is that amount sufficient to achieve a medically desirable result.

Generally, doses of active compounds of the present invention would be from about 0.01 mg/kg per day to 1000 mg/kg per day. It is expected that doses ranging from 50-500 mg/kg will be suitable. A variety of administration routes are available. The methods of the invention, generally speaking, may be practiced using any mode of administration that is medically acceptable, meaning any mode that produces effective levels of the active compounds without causing clinically unacceptable adverse effects. Such modes of administration include oral, rectal, topical, nasal, interdermal, or parenteral routes. In some embodiments of the invention, the mode of administration is direct injection into the thyroid tissue. The term “parenteral” includes subcutaneous, intravenous, intramuscular, or infusion. Intravenous or intramuscular routes are not particularly suitable for long-term therapy and prophylaxis. They could, however, be preferred in emergency situations. Oral administration will be preferred for prophylactic treatment because of the convenience to the patient as well as the dosing schedule. Techniques for preparing aerosol delivery systems are well known to those of skill in the art. Generally, such systems should utilize components which will not significantly impair the biological properties of the active ingredients (see, for example, Sciarra and Cutie, “Aerosols,” in Remington's Pharmaceutical Sciences, 18th edition, 1990, pp 1694-1712; incorporated by reference).

Compositions suitable for oral administration may be presented as discrete units, such as capsules, tablets, lozenges, each containing a predetermined amount of the active agent. Other compositions include suspensions in aqueous liquids or non-aqueous liquids such as a syrup, elixir or an emulsion.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compounds.

CD1 fusion proteins and complexes thereof may be combined, optionally, with a pharmaceutically-acceptable carrier. The term “pharmaceutically-acceptable carrier” as used herein means one or more compatible solid or liquid filler, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

When administered, the pharmaceutical preparations of the invention are applied in pharmaceutically-acceptable amounts and in pharmaceutically-acceptably compositions. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Other delivery systems can include time-release, delayed release or sustained release delivery systems. Such systems can avoid repeated administrations increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

Use of a long-term sustained release implant may be particularly suitable for treatment of chronic conditions. Long-term release, are used herein, means that the implant is constructed and arranged to delivery therapeutic levels of the active ingredient for at least 30 days, and preferably 60 days. Long-term sustained release implants are well-known to those of ordinary skill in the art and include some of the release systems described above.

A variety of other reagents also can be included in the binding mixture. These include reagents such as salts, buffers, neutral proteins (e.g., albumin), detergents, etc. which may be used to facilitate optimal protein-protein interactions. Such a reagent may also reduce non-specific or background interactions of the reaction components. Other reagents that improve the efficiency of the assay may also be used. The mixture of the foregoing assay materials is incubated under conditions under which the CD1 fusion protein normally specifically binds to its CD1 antigen. Such conditions have been previously disclosed in both patents and patent applications cited herein. The order of addition of components, incubation temperature, time of incubation, and other parameters of the assay may be readily determined. Such experimentation merely involves optimization of the assay parameters, not the fundamental composition of the assay. Incubation temperatures typically are between 4° C. and 40° C. Incubation times preferably are minimized to facilitate rapid, high throughput screening, and typically are between 0.1 and 10 hours. After incubation, the presence or absence of specific binding between the CD1 fusion protein and the library molecule, for example, is detected by any convenient method available to the user.

Typically, a plurality of assay mixtures are run in parallel with different agent concentrations to obtain a different response to the various concentrations. One of these concentrations serves as a negative control, i.e., at zero concentration of agent or at a concentration of agent below the limits of assay detection.

For cell-free binding type assays, a separation step is often used to separate bound from unbound components. The separation step may be accomplished in a variety of ways. Conveniently, at least one of the components is immobilized on a solid substrate, from which the unbound components may be easily separated. The solid substrate can be made of a wide variety of materials and in a wide variety of shapes, e.g., columns or gels of polyacrylamide, agarose or sepharose, microtiter plates, microbeads, resin particles, etc. The substrate preferably is chosen to maximum signal to noise ratios, primarily to minimize background binding. The separation step preferably includes multiple rinses or washes. For example, when the solid substrate is a microtiter plate, the wells may be washed several times with a washing solution, which typically includes those components of the incubation mixture that do not participate in specific bindings such as salts, buffer, detergent, non-specific protein, etc. Where the solid substrate is a magnetic bead, the beads may be washed one or more times with a washing solution and isolated using a magnet.

For cell-free binding assays, one of the components usually comprises, or is coupled to, a detectable label. A wide variety of labels can be used, such as those that provide direct detection (e.g., radioactivity, luminescence, optical or electron density, etc.) or indirect detection (e.g., epitope tag such as the FLAG epitope, enzyme tag such as horseradish peroxidase, etc.). The label may be bound to a library member, or incorporated into the structure of the library member. CD1 fusion proteins and/or CD1 antigens may also be labeled by a variety of means for use in screening assays or diagnostic assays. There are many different labels and methods of labeling known to those of ordinary skill in the art. Examples of the types of labels which can be used in the present invention include enzymes, radioisotopes, fluorescent compounds, colloidal metals, chemiluminescent compounds, and bioluminescent compounds. Those of ordinary skill in the art will know of other suitable labels for binding to the binding partners used in the screening assays, or will be able to ascertain such, using routine experimentation. Furthermore, the coupling of these labels to the binding partners used in the screening assays of the invention can be done using standard techniques common to those of ordinary skill in the art.

Another labeling technique which may result in greater sensitivity consists of coupling the binding partners to low molecular weight haptens. These haptens can then be specifically altered by means of a second reaction. For example, it is common to use haptens such as biotin, which reacts with avidin, or dinitrophenol, pyridoxal, or fluorescein, which can react with specific anti-hapten antibodies.

A variety of methods may be used to detect the label, depending on the nature of the label and other assay components. For example, the label may be detected while bound to the solid substrate or subsequent to separation from the solid substrate. Labels may be directly detected through optical or electron density, radioactive emissions, nonradiative energy transfers, etc. or indirectly detected with antibody conjugates, streptavidin-biotin conjugates, etc. Methods for detecting the labels are well known in the art.

### EXAMPLES

Introduction to the Examples:

An illustrative procedure for making and using a CD1d fusion protein is provided in the Examples. It is to be understood that the methods disclosed herein are representative of methods for making the claimed compositions and that alternative methods for making the CD1d and other CD1 fusion proteins can be substituted for the instant methods without departing from the essence of the invention. To generate similar b2m-linked CD1-Fc fusion proteins, nucleotides 403-1239 of the murine CD1d-Fc construct described below and in Gumperz, J. E. et al., Immunology, 12:211-221 (Feb. 2000) would be substituted with the corresponding regions of cDNA encoding either human CD1a (genbank accession #M28825, Seq. ID No.1), CD1b (genbank accession #M28826, Seq. ID No. 2), CD1c (genbank accession #M28827, Seq. ID No: 3), CD1d (genbank accession #X14974, Seq. ID No: 4), or CD1e (genbank accession #X14975, Seq. ID No. 5).

A brief summary of the instant methods is provided below.

To make the fusion proteins of the invention, new cDNA constructs were generated that encode human beta-2 microglobulin attached by a glycine-serine spacer peptide to the N-terminus of the extracellular domains of CD1. The C-terminus of the CD1 molecule is fused by another glycine-serine spacer peptide to the hinge and CH—CH3 domains of murine IgG₂ₐ. The cDNA constructs were cloned into the pBJ1-neo expression vector, for stable expression in mammalian cells (Lin, A. et al., Science, 249:677-679 (1990)). The fusion proteins were expressed in CHO cells, and purified from the culture supernatant using a protein A affinity column and pH 4.3 acid buffer elution. Analysis by SDS-PAGE and size exclusion chromatography indicate the fusion proteins are secreted as glycosylated, disulfide-linked dimers of the expected molecular weight of aproximately 200 kD. Using a standard double antibody sandwich ELISA technique, the fusion proteins were detected with monoclonal antibody (mAb) specific for the native CD1d molecules, human beta-2microglobulin, and murine IgG₂ₐ.

The fusion proteins can be coated on plastic and used to investigate the functional reactivity of CD1-restricted T cells to specific lipid antigens, as shown in the Examples.

To facilitate binding to CD1 specific T cells for detection by flow cytometry, a highly multimerized form of the CD1d fusion protein was formed using fluorescently labeled protein A molecules. Protein A molecules spontaneously associate in solution at neutral pH with immunoglobulin Fc regions, forming complexes containing four Fc molecules and two protein A molecules (4+2 complexes, reference 2). The human CD1d-Fc fusion protein was incubated with Alexa 488 dye labeled protein A, and the 4+2 complexes purified by size exclusion chromatography on a Pharmacia Superose 6 column using PBS pH 7.2 as a running buffer. The purified 4+2 aggregates were concentrated to 100 μg/ml with ovalbumin as a carrier protein. The CD1d-Fc aggregate was then pre-incubated for 24 to 48 hours at 37° C. with antigenic glycolipids dissolved in DMSO at a 40:1 molar ratio of lipid to fusion protein, or with an equivalent volume of DMSO alone as a negative control. The T cell staining was performed at room temperature or 4° C. for 20 min, at a concentration of 40 μg/ml of the lipid or control treated CD1d-Fc aggregate.

To test the specificity of staining, previously isolated human CD1d-restricted T cell clones (Spada, F. M., et al., J. Exp. Med. 188(8):1529-34.1 (1998)) were stained with Cd1D-Fc aggregates treated with lipid antigens or control compounds. Flow cytometric analysis showed that the CD1d fusion protein aggregates treated with specific lipid antigens such as α-galactosyl ceramide (α-GalCer), and α-glucosyl ceramide (α-GlcCer) gave positive staining, whereas the CD1d-Fc aggregates treated with the related lipids α-mannosyl ceramide (α-ManCer), β-galactosyl ceramide (β-GalCer), ceramide (Cer), or DMSO alone did not stain above background levels (see Example figures). This experiment demonstrates the requirement for treatment of the CD1d fusion protein with specific lipid antigens to enable stable binding to “cognate T cells.” Furthermore, the lipid antigen specificity in these staining experiments correlated precisely with the functional reactivity to lipid antigens presented by CD1d molecules previously observed for these T cell clones (Kawano, T. et al., Science, 278(5343):1626-9 (1997); Spada, F. M. et al, J. Exp. Med., 188:1529-34 (1998)). The specificity of staining was further confirmed by comparing staining of 2 CD1d-restricted T cell clones with that of 4 T cell clones that are not CD1d-restricted. The lipid antigen treated fusion protein positively stains the CD1d-restricted T cells, but does not stain the non-CD1d-restricted T cells above background levels (see Example figures).

To investigate whether the lipid loaded fusion protein can detect CD1d reactive T cells in peripheral blood, three color flow cytometric analysis was performed on PBMCs purified from a healthy donor. The cells were stained with anti-CD3, anti-CD161, and the -GalCer antigen loaded or DMSO treated CD1d-Fc aggregates, or an aggregate made with a negative control antibody (UPC10). The CD1d-Fc aggregate treated with α-GalCer stained about 6-fold as many T cells as the CD1d-Fc treated with DMSO alone, and about 10-fold as many as the UPC10 negative control. A population of CD3⁻ lymphocytes was stained by all three protein A aggregated reagents, suggesting this was due to non-specific binding. However, very few CD3⁺ cells were stained by the negative control UPC10 complex, indicating very low non-specific binding of this type of staining reagent to T cells. This experiment suggests that this reagent can be used to detect lipid antigen specific CD1d-restricted T cells directly in peripheral blood samples.

T cell lines and clones stained with the α-GalCer treated CD1d-Fc aggregates were isolated from peripheral blood flow cytometric cell sorting and limiting dilution cloning, and cultured using standard techniques. Functional analysis of the T cell lines and clones revealed that they secrete cytokines in response to CD1d-transfected antigen presenting cells, but not to the untransfected parent cells. This experiment shows that T cells isolated using the α-GalCer treated Cd1d-Fc fusion protein are CD1d-restricted, and can recognize CD1d molecules at the cell surface of antigen presenting cells that may be complexed with endogenous lipid antigens, and that the T cells also respond strongly to the α-GalCer lipid antigen.

### Example 1

### Murine CD1d-Restricted T Cell Recognition of Cellular Lipids

NKT cells are associated with immunological control of autoimmune disease and cancer, and can recognize cell surface mCD1d without addition of exogenous antigens. Cellular antigens presented by mCD1d have not been identified, although NKT cells can recognize a synthetic glycolipid, α-GalCer. Here we show that after addition of a lipid extract from a tumor cell line, plate-bound mCD1d molecules stimulated an NKT cell hybridoma. This hybridoma also responded strongly to three purified phospholipids, but failed to recognize α-GalCer. Seven of 16 other mCD1d-restricted hybridomas also showed a response to certain purified phospholipids. These findings suggest NKT cells can recognize cellular antigens distinct from α-GalCer, and identify phospholipids as potential self antigens presented by mCD1d.

CD1 molecules are evolutionarily conserved β₂-microglobulin (β₂m) associated proteins, with a similar domain organization to class I antigen presenting molecules of the major histocompatibility complex (Porcelli, S. A., Adv. Immunol., 59:1-98 (1995)). However, CD1 molecules have a deeper and more hydrophobic antigen binding groove than class I molecules (Zeng, Z. -H. et al., Science, 277:339-45 (1997)). Correspondingly, while class I molecules present peptide antigens, CD1 molecules can present lipids and glycolipids. Studies of human CD1a, b, and c molecules first demonstrated they can present microbial glycolipid antigens to T cells (Beckman, E. M. et al., J. Immunol., 157:2795-803 (1996); Beckman, E. M. et al., Nature, 372:691-4 (1994); Sieling, P. A. et al., Science, 269:227-30 (1995)). Subsequently, both human and murine CD1d molecules have been shown to present α-galactosylcerarnide (α-GalCer), a synthetic acylphytosphingolipid originally isolated from a marine sponge (Kawano, T. et al., Science, 278:1626-9 (1997)); Spada, F. M. et al, J. Exp. Med., 188:1529-34 (1998)).

The T cells that recognize murine CD1d molecules are either CD4⁺, or negative for both CD4 and CD8β (double negative, or “DN”) (Bendelac, A. et al., Science, 263:1774-8 (1994); Bendelac, A. et al., Science, 268:863-5 (1995)). At least two distinct populations of CD1d-restricted αβ T cells have been identified in the mouse, based on their T cell receptor (TCR) structures. One population has a characteristic invariant TCRα chain (Vα14/Jα281) paired preferentially with TCR β chains utilizing Vβ8. These cells comprise a part of the NKT cell subset, T cells that express receptors of the NK complex (Lantz, O., and Bendelac, A., J. Exp. Med., 180:1097-106 (1994); Taniguchi, M. et al, PNAS, 93:11025-8 (1996)). More recently, T cells expressing diverse TCR α and β chains have also been found that recognize mCD1d molecules (Behar, S. M. et al., J. Immunol., 162:161-7 (1999); Cardell, S. et al., J. Exp. Med., 182:993-1004 (1995); Chiu, Y. H. et al., J. Exp. Med., 189:103-10 (1999)). Similar to those of the “NKT” subset, CD1d-restricted cells belonging to this “diverse TCR” population can secrete significant amounts of IL-4 and IL-10 in addition to IFNγ, and may thus contribute to determining the TH₁/TH₂ cytokine balance in immune responses (Behar, S. M. et al., J. Immunol., 162:161-7 (1999); Yoshimoto, T. et al, Science, 270:1845-7 (1995)). CD1d-restricted T cells have also been associated with various immunologically mediated functions, such as preventing development of autoimmune diabetes, tumor rejection, and modulating IgG responses during protozoal infections (Chiu, Y. H. et al., J. Exp. Med., 189:103-10 (1999); Schofield, L. et al., Science, 283:225-9 (1999); Wilson, S. B. et al., Nature, 391:177-81 (1998)).

The origin and the identity of the natural antigens recognized by CD1d-restricted T cells remain unknown. It has been postulated that mCD1d-restricted NKT cells may recognize a single or a conserved set of antigens, since their cannonical α chains and limited β chain diversity result in TCRs of comparatively little structural variability, whereas the diverse TCR population of mCD1d-restricted T cells may have heterogeneous antigenic specificities (Behar, S. M. et al., J. Immunol., 162:161-7 (1999); Cardell, S. et al., J. Exp. Med., 182:993-1004 (1995); Chiu, Y. H. et al., J. Exp. Med., 189:103-10 (1999)). Both T cell populations can recognize CD1d molecules on antigen presenting cells (APCs) in vitro, without requiring addition of exogenous antigens (Behar, S. M. et al., J. Immunol., 162:161-7 (1999); Bendelac, A. et al., Science, 268:863-5 (1995)). Whether this phenomenon is due to recognition of the CD1d heavy chain itself, or represents recognition of CD1d complexed with cellular antigens or exogenous antigens derived from the culture medium, is unclear. NKT cells have also been shown to respond to synthetic α-GalCer in a CD1d dependent manner, but this antigen has thus far not been found in mammalian tissues (Kawano, T. et al., Science, 278:1626-9 (1997)). Hence, neither the nature of the cellular antigens bound by CD1d molecules, nor whether these antigens are required for T cell recognition of CD1d molecules, is well understood.

Here, we investigated the requirement for presentation of cellular antigens in T cell recognition of mCD1d molecules, and examined the antigen specificities of mCD1d-restricted T cells of the NKT cell and diverse TCR populations. We developed a system to study recognition of mammalian lipids, using an immobilized murine CD1d fusion protein and purified antigen preparations. Recognition of the recombinant mCD1d fusion protein in this system was dependent on the addition of particular lipids, permitting analysis of the lipid antigen specificities of mCD1d-restricted T cells. Our results provide evidence that mCD1d-restricted T cells require presentation of specific antigens for recognition of mCD1d molecules. Surprisingly, our findings suggest the mCD1d-restricted NKT cell subset surveys multiple cellular antigens distinct from α-GalCer, and implicate common phospholipids as potential autoantigens recognized by certain NKT cells.

An mCD1d-restricted NKT Cell Hybridoma Responds to a Lipid Extract from RMA-S Cells

Certain mCD1d-restricted T cells do not require exogenous antigens for mCD1d recognition, suggesting they may recognize mCD1d molecules directly, or may recognize cellular antigens complexed with mCD1d (Behar, S. M. et al., J. Immunol., 162:161-7 (1999); Bendelac, A. et al., Science, 268:863-5 (1995)). To investigate whether cellular lipids are involved in such recognition, we studied an NKT cell clone, called 24.8, which recognizes mCD1d expressed on murine splenocytes and dendritic cells, as well as on mCD1d transfected RMA-S tumor cells (Behar, S. M. et al., J. Immunol., 162:161-7 (1999), and S.M.B. unpublished observations). Because hybridomas can produce IL-2 in response to antigenic stimulation in the absence of additional co-stimulatory signals, a T cell hybridoma, designated 24.8.A, was derived from this clone.

To investigate mCD1d recognition by the 24.8.A hybridoma, we tested a soluble mCD1d-IgGFc₂ₐ fusion protein which had been purified and immobilized on protein A coated plates, for its ability to stimulate IL-2 release. The 24.8.A hybridoma usually secreted a modest amount of IL-2 when incubated with the mCD1d fusion protein (50-300 pg/ml in 60% of the experiments), but occasionally produced high levels of IL-2 (>600 pg/ml in 20% of the experiments), or did not generate quantifiable IL-2 (20% of the experiments). In contrast, incubation with an immobilized anti-CD3 mAb consistently resulted in very high levels of IL-2 secretion (usually >2000 pg/ml IL-2). No detectable IL-2 was secreted when the 24.8.A hybridoma was incubated with a negative control protein (IgG₂ₐ mAb RPC5.4 or UPC10) immobilized on the protein A plate.

The poor stimulation of the 24.8.A hybridoma by the mCD1d fusion protein suggested that a specific cellular antigen might be required for efficient recognition of the recombinant mCD1d molecule. We reasoned that an appropriate antigen should be contained within a lipid extract made from RMA-S cells, since these cells can be efficiently recognized when they are transfected with mCD1d (Behar, S. M. et al., J. Immunol., 162:161-7 (1999)). A modified Folch extraction protocol was used to purify biochemical fractions from RMA-S and S49 T lymphoma cells (Folch, J. et al., J. Biol. Chem., 226:497-509 (1956); Hamilton, S. et al., Oxford: IRL Press at Oxford University (1992)). The resulting aqueous, organic, and interface fractions were tested for the ability to stimulate the 24.8.A hybridoma. Plate-bound mCD1d fusion protein or the negative control protein were pre-incubated with the cellular fractions, then repeatedly washed to remove unbound material prior to addition of the 24.8.A hybridoma. Pre-treatment of the mCD1d fusion protein with the organic phase of the RMA-S extract resulted in markedly augmented IL-2 release by the 24.8.A hybridoma compared to the mCD1d fusion protein treated with buffer. In contrast, the mCD1d fusion protein pre-incubated with the interface induced only a small increase in IL-2 production, and treatment with the aqueous phase did not enhance IL-2 secretion compared to the buffer treated control. The negative control protein failed to induce significant IL-2 secretion, when pre-incubated with any of the Folch fractions. Thus, stimulation was dependent on the presence of the mCD1d fusion protein, and specific for the organic phase of the cellular extract, which contains mainly the cellular lipids (Folch, J. et al., J. Biol. Chem., 226:497-509 (1956); Hamilton, S. et al., Oxford. IRL Press at Oxford University (1992)).

To examine further the antigen dependence of the hybridoma, the amount of organic extract added to the plate-bound mCD1d fusion protein was titrated. Titration of the lipid extract from 0.03 μg/well to 10 μg/well, produced a dose dependent response which appeared saturated at 1 μg/well. In the presence of a negative control anti-MHC class II mAb the titration curve was nearly identical, but an anti-mCD1d blocking mAb completely abrogated the response. Organic extracts from S49 cells gave similar results. Hence, the lipid fraction of mammalian cellular extracts contained antigenic material, that stimulated the 24.8.A hybridoma in an mCD1d and dose-dependent manner.

To characterize the nature of the antigen contained in the cellular lipid extract, the organic phase preparations from the Folch extractions were further fractionated using a silica column. Lipids of increasing polarity were eluted sequentially from the column with chloroform, acetone, and methanol, resulting in separation of fractions that predominantly contained neutral lipids, glycolipids, and phospholipids respectively. These fractions were tested for stimulation of the 24.8.A hybridoma, compared to the unfractionated organic phase of the extract, by titrating the amount of each fraction pre-incubated with the plate-bound mCD1d fusion protein. Addition of the chloroform fraction did not induce detectable IL-2 production. In contrast, pre-treatment of the mCD1d fusion protein with the acetone and methanol fractions resulted in dose-dependent stimulation of the 24.8.A hybridoma. Hence, the 24.8.A hybridoma recognized fractions of the organic extract containing polar lipids, but did not respond to a fraction enriched in neutral lipids.

Recognition of Synthetic Antigens by NKT Cell Hybridomas

The acylphytosphingolipid, α-GalCer, and glycosylated phosphatidylinositols are lipid antigens thought to bind and be presented by mCD1d molecules (Joyce, S. et al., Science, 279:1541-4 (1998); Kawano, T. et al., Science, 278:1626-9 (1997); Schofield, L. et al., Science, 283:225-9 (1999)). Our finding that addition of cellular organic extracts containing polar lipids permitted efficient recognition of the mCD1d fusion protein, suggested the 24.8.A hybridoma recognizes an abundant mammalian lipid. To investigate recognition of potential cellular lipid antigens, we tested a purified preparation of the phospholipid phosphatidylinositol (PI), and a series of purified and synthetic sphingolipids, for recognition by the 24.8.A hybridoma, and by another NKT cell hybridoma, called 24.9.E. Plate-bound mCD1d fusion protein or a negative control protein were pre-treated with α-GalCer, β-GalCer, unglycosylated ceramide, the naturally occurring gangliotriosyl-ceramide (asialo-GM₂), and PI, prior to addition of the hybridomas. The 24.8.A hybridoma showed only a slightly enhanced response to the mCD1d fusion protein which had been pre-incubated with the α-GalCer antigen or the other sphingolipids, compared to untreated fusion protein. However, pre-treatment of the mCD1d fusion protein with PI resulted in a marked increase of IL-2 production. In contrast, the 24.9.E hybridoma responded strongly to the mCD1d fusion protein which had been pre-incubated with α-GalCer, but showed only modestly increased IL-2 secretion in response to the PI treated mCD1d fusion protein. Consistent with the results of Kawano et al., stimulation of the 24.9.E NKT cell hybridoma required the α-linked galactose to be present on the galactosylceramide antigen, since neither the unglycosylated ceramide, nor the closely related β-linked form, β-GalCer, were recognized. The asialo-GM₂ sphingolipid also was not recognized. Pre-treatment of the negative control protein with any of the lipids failed to induce detectable IL-2 secretion by either hybridoma. Thus, while the 24.8.A. and 24.9.E hybridomas both required addition of a lipid antigen to the mCD1d fusion protein for efficient activation, they appeared to have distinct antigen specificities.

Titration of the molar ratio of antigen to fusion protein from 10:1 to 80:1 confirmed the antigen specific, dose-dependent responses of the 24.8.A and 24.9.E hybridomas. IL-2 production by the 24.8.A hybridoma appeared saturated at a 40:1 molar ratio of PI to mCD1d fusion protein, while little IL-2 was secreted even at an 80:1 molar excess of α-GalCer. In contrast, the 24.9.E hybridoma secreted IL-2 efficiently in response to α-GalCer treated mCD1d fusion protein, but generated significantly less IL-2 even at high ratios of PI to mCD1d. To confirm that this antigen dependent stimulation of the NKT cell hybridomas was mCD1d specific, the 19G11 anti-mCD1d blocking antibody was used. In a representative experiment, the 24.8.A hybridoma secreted a mean of 4,746 pg/ml IL-2 in response to mCD1d fusion protein pre-treated with PI, but in the presence of the 19G11 mAb no detectable IL-2 was produced. For the 24.9.E hybridoma pre-treatment with α-GalCer resulted in production of a mean of 2,089 pg/ml IL-2, which was reduced to 103 pg/ml when the 19G11 anti-mCD1d mAb was included. Hence, a antigen specific activation of the hybridomas by the mCD1d fusion protein could be blocked by addition of an anti-mCD1d antibody.

Specificity of Phospholipid Antigen Recognition

To examine the specificity of PI recognition by the 24.8.A hybridoma, analogues of PI were tested with the mCD1d fusion protein. Three synthetic PIs with one, two, or three additional phosphate groups attached to carbons of the inositol ring (PI3-P, PI3, 4-P2, and PI3, 4, 5-P3, respectively) were compared to PI, and to successively smaller constituent components of PI: phosphatidic acid (PA) which lacks the inositol ring of PI, diacyl glycerol (DAG) which lacks the phosphate of PA, palmitic acid which corresponds to one free acyl chain of the DAG molecule, and free inositol. As previously observed, pre-treatment of the mCD1d fusion protein with PI resulted in significantly enhanced IL-2 release. Treatment of the fusion protein with components of PI lacking the inositol ring attached to the acyl chains, (PA, DAG, palmitate, and inositol), provided little or no stimulation. IL-2 secretion induced by pre-treatment with the synthetic phosphorylated PI antigens was also significantly greater than that for the mCD1d fusion protein incubated with buffer alone. These results suggested the inositol ring was an important antigenic determinant of PI for the 24.8.A hybridoma.

To confirm the importance of the inositol ring in recognition of PI, the PI was phospholipase treated prior to incubation with the fusion protein. Two different phospholipases were tested. Phospholipase D (PLD) removes the inositol ring from the phosphate which links it to the diacyl glycerol backbone, to yield free inositol and phosphatidic acid (PA). PI-specific phospholipase C (PI-PLC) cleaves the bond between the phosphate and the glycerol, to produce inositol phosphate and diacyl glycerol (DAG). Treatment of PI with PLC or PLD prior to pre-incubation of the antigen with the mCD1d fusion protein reduced IL-2 secretion by approximately 70%, approaching the IL-2 levels seen when synthetic preparations of DAG or PA were incubated with the fusion protein. Thus, the inositol ring appears to be important for PI recognition by the 24.8.A hybridoma in this system, and forms of PI which are phosphorylated on the inositol ring can also be recognized.

We next examined the specificity of the 24.8. A hybridoma for PI compared to other common phospholipid antigens. Four additional phospholipids related to PI were tested: phosphatidylcholine (PC), phosphatidylethanolamine (PE), phosphatidylglycerol (PG), and phosphatidylserine (PS). Titrations of the molar ratio of antigen to fusion protein from 10:1 to 80:1 were carried out for these antigens. The 24.8.A hybridoma demonstrated dose dependent responses to the PE and PG antigens, which appeared saturated at a molar ratio of 40:1 antigen to fusion protein. Pre-incubation with PC or PS did not reproducibly significantly enhance reactivity to the mCD1d fusion protein. Hence, recognition of the mCD1d fusion protein by the 24.8.A hybridoma was clearly augmented by pre-treatment with PI, PE, and PG, but not with PS or PC. Taken together these results are consistent with a model in which the acyl chains of the lipid tails are required for binding to CD1 molecules, but antigen specificity is determined by TCR recognition of features of the polar head group (Porcelli, S. A., and Brenner, M. B., Current Biology, 7(8):R508-11 (1997)).

The Effect of pH on Antigen Recognition

Previous studies have suggested that CD1d molecules may encounter antigens in intracellular vesicles that undergo substantial acidification during the process of antigen loading (Brossay, L. et al, J. Immunol., 160:3681-8 (1998); Chiu, Y. H. et al., J. Exp. Med., 189:103-10 (1999); Kawano, T. et al., Science, 278:1626-9 (1997); Spada, F. M. et al, J. Exp. Med., 188:1529-34 (1998)). The 24.9.E hybridoma was used to examine the effect of acidic pH on α-GalCer presentation by the mCD1d fusion protein. The mCD1d fusion protein was incubated with α-GalCer antigen diluted into citrate/phosphate buffer solutions ranging from pH 7.5 to pH 3.0, at a 3:1 molar ratio of antigen to protein, then the solutions were neutralized to allow binding to the protein A coated plate, and assayed for recognition by the 24.9.E hybridoma. Recognition of the α-GalCer antigen was enhanced approximately 4 fold after antigen pre-incubation at pH 4.0, compared to pH 7.5. Maximal IL-2 release was reproducibly observed for the samples pre-incubated at pH 4.0, while IL-2 production dropped significantly for samples pre-incubated below this pH. Negative control wells containing the mCD1d fusion protein diluted into the pH titrated citrate/phosphate buffer solutions with no antigen added, or a negative control protein treated with α-GalCer at pH 7.2, did not induce detectable IL-2 production. To ensure that pre-incubation at low pH did not affect binding of the fusion protein to the protein A plate, the assay plate was tested (after removal of the culture supernatants) for the presence of mCD1d using a biotinylated rat anti-mCD1d mAb(19G11) which does not bind to protein A, followed by detection with a streptavidin-enzyme conjugate and a chromogenic substrate. This analysis revealed that the amount of mCD1d fusion protein bound to the plate was not affected by the pre-incubation pH. Therefore, although antigens incubated at physiological pH could be recognized, treatment of the mCD1d fusion protein with α-GalCer at pH 4.0 provided optimal antigen recognition in this system.

Comparison of Antigen Recognition By Diverse And NKT Cell mCD1d-restricted Hybridomas

Our observation that two NKT cell hybridomas, 24.8.A and 24.9.E, differed in their antigen reactivity, raised the possibility that NKT cells may have heterogeneous antigen specificities. To extend our analysis of NKT cells, and to compare antigen recognition by mCD1d-restricted T cells of the diverse TCR population, we tested 9 NKT and 8 diverse TCR mCD1d-restricted hybridomas for recognition of 14 purified and synthetic lipid antigens (see Tables 1 and 2). None of the hybridomas produced detectable IL-2 in response to a negative control protein, and only the 24.8.A hybridoma secreted detectable IL-2 in response to untreated mCD1d fusion protein (Table 2). Eight out of nine NKT cell hybridomas were potently stimulated by α-GalCer treated fusion protein, whereas none of the diverse TCR hybridomas reproducibly recognized this antigen (Table 2). Purified PI strongly stimulated the 24.8.A hybridoma, and also stimulated some of the -GalCer reactive NKT cell hybridomas, although with only about 10-20% of the activity of the synthetic α-GalCer. Several diverse TCR hybridomas also secreted detectable IL-2 upon incubation with PI, PE, or PG treated mCD1d fusion protein (Table 2). None of the hybridomas reproducibly recognized any of the other antigens tested. Thus, in this antigen screen most (8/9) of the NKT cell hybridomas recognized α-GalCer, whereas all but one of the diverse TCR hybridomas failed to respond to this antigen. In contrast, approximately half of both the NKT and diverse TCR hybridomas tested showed some reactivity to certain phospholipid antigens.

TABLE 1
TCR Gene Usage of TT Hybridoma Cells Used for Analysis

| Hybridoma | Lineage | Vα/Jα Genes | Vα/Jα Genes |
|---|---|---|---|
| 24.8.A | NKT | Vα14/Ja281 | Vβ8.2/Jβ2.5 |
| 24.7.C | NKT | Vα14/Ja281 | Vβ6.1/Jβ2.6 |
| 24.9.E | NKT | Vα14/Ja281 | Vβ8.3/Jβ2.4 |
| DN32D3 | NKT | Vα14/Ja281 | Vβ8.2/Jβ2.4 |
| KT/7 | NKT | Vα14/Ja281 | Vβ8.2/ND |
| KT/12 | NKT | Vα14/Ja281 | Vβ8.2/ND |
| KT/22 | NKT | Vα14/Ja281 | Vβ8.2/ND |
| KT/23 | NKT | Vα14/Ja281 | Vβ8.2/ND |
| Vβ/9 | NKT | Vα14/Ja281 | Vβ8.2/ND |
| 14S.6.A | diverse | Vα17.1/JαTT11 | Vβ14.1/Jβ2.1 |
| 14S.7.N | diverse | Vα15.1/JαNEW.02 | Vβ8.2/Jβ2.5 |
| 14S.10.C | diverse | Vα11.3/JαNEW.15 | Vβ8.1/Jβ2.6 |
| 14S.15.A | diverse | Vα10.2/9/JaTA65 | Vβ5.1/Jβ2.4 |
| VII68 | diverse | Vα4/Jα25 | Vβ11/Jβ2.5 |
| VIII24 | diverse | Vα3.2/Jα20 | Vβ9/Jβ1.4 |
| XV19 | diverse | ND | ND |
| XV104 | diverse | Vα4/5/ND | Vβ8.3/Jβ2.6 |

TCR α and β gene usage for the 24.7.C, 24.8.A, 24.9.E, DN32D3, 14S.6.A, 14S.7.N, 14S.10.C, 14S.15.A, VII68, VIII24, and XV104 hybridomas was determined by DNA sequencing. For the KT/7, KT/12, KT/22, KT/23, and Vβ/9 hybridomas, the presence of the Vα14/Jα281 rearranged TCR α chain was determined by PCR analysis, and the Vβ chain usage was assessed by flow cytometry.

TABLE 2
mCD1-Restricted Hybridoma Responses to Plate-Bound mCD1d
Fusion Protein Preincubated with Lipid Antigens

| | Invariant TCRα NKT Hybridomas | | | | | | | | |
|---|---|---|---|---|---|---|---|---|---|
| | 24.8 A | 24 7.C | 24.9 E | DN32D3 | KT/7 | KT/12 | KT/22 | KT/23 | Vβ/9 |
| No mCD1d | 0 | 0 | 0 | 0 | | | | | |
| No Ag | + | 0 | 0 | 0 | 0 | 0 | 0 | 0 | 0 |
| α-GalCer | + | +++ | +++ | +++ | +++ | +++ | +++ | +++ | ++ |
| β-GalCer | + | 0 | 0 | 0 | 0 | 0 | 0 | 0 | 0 |
| Cer | + | 0 | 0 | 0 | 0 | 0 | 0 | 0 | 0 |
| Sph | + | 0 | 0 | 0 | 0 | 0 | 0 | 0 | 0 |
| aGM₂ | + | 0 | 0 | 0 | 0 | 0 | 0 | 0 | 0 |
| GD1a | 0 | 0 | 0 | 0 | 0 | 0 | 0 | 0 | 0 |
| PA | + | 0 | 0 | 0 | 0 | 0 | 0 | 0 | 0 |
| PI | +++ | 0 | + | 0 | 0 | + | + | + | 0 |
| PS | + | 0 | 0 | 0 | 0 | 0 | 0 | 0 | 0 |
| PG | ++ | 0 | + | 0 | 0 | 0 | 0 | 0 | 0 |
| PE | | 0 | 0 | 0 | | | | | |
| PC | + | 0 | 0 | 0 | 0 | 0 | 0 | 0 | 0 |
| MGDG | + | 0 | 0 | 0 | 0 | 0 | 0 | 0 | 0 |
| DAG | | 0 | 0 | 0 | 0 | 0 | 0 | 0 | 0 |

| | Diverse TCR Hybridomas | | | | | | | |
|---|---|---|---|---|---|---|---|---|
| | 14S.6.A | 14S.7.N | 14S.10 C | 14S.15.A | VII68 | VIII24 | XV19 | XV104 |
| No mCD1d | 0 | 0 | 0 | 0 | | | | |
| No Ag | 0 | 0 | 0 | 0 | 0 | 0 | 0 | 0 |
| α-GalCer | 0 | 0 | 0 | + | 0 | 0 | 0 | 0 |
| β-GalCer | 0 | 0 | | 0 | 0 | 0 | 0 | 0 |
| Cer | 0 | 0 | 0 | 0 | 0 | 0 | 0 | 0 |
| Sph | 0 | 0 | 0 | + | 0 | 0 | 0 | 0 |
| aGM₂ | 0 | 0 | 0 | 0 | 0 | 0 | 0 | 0 |
| GD1a | 0 | 0 | 0 | 0 | 0 | 0 | 0 | 0 |
| PA | 0 | 0 | | 0 | 0 | 0 | 0 | 0 |
| PI | + | + | 0 | 0 | 0 | 0 | 0 | 0 |
| PS | 0 | 0 | 0 | 0 | 0 | 0 | 0 | 0 |
| PG | + | + | | + | 0 | 0 | 0 | 0 |
| PE | + | 0 | | 0 | | | | |
| PC | 0 | 0 | 0 | 0 | 0 | 0 | 0 | 0 |
| MGDG | 0 | 0 | | 0 | 0 | 0 | 0 | 0 |
| DAG | 0 | 0 | | 0 | 0 | 0 | 0 | 0 |
| IL-2 secretion by mCD1d-restricted hybridomas in response to plate-bound mCD1d-IgGFc₂ₐ fusion protein and lipid antigens. A “0” indicates a mean of less than 50 pg/ml IL-2 was secreted, “+” indicates 50-250 pg/ml, “++” indicates 250-1000 pg/ml, “+++” indicates greater than 1000 pg/ml IL-2 | | | | | | | | |
| #secretion, spaces left blank were not done in the experiment shown. Negative control wells contained neither fusion protein nor antigen (No mCD1d). The mCD1d fusion protein was pre-incubated with buffer alone (No Ag); α-galactosylceramide (α-GalCer); β-galactosylceramide (β-GalCer); unglycosylated ceramide (Cer); sphingomyelin (Sph); | | | | | | | | |
| #gangliotriosyl ceramide (aGM₂); disialoganglioside (GD1a); phosphatidic acid (PA); phosphatidylinositol (PI); phosphatidylserine (PS); phosphatidylglycerol (PG); phosphatidylethanolamine (PE); phosphatidylcholine (PC); monogalactosyl diglyceride (MGDG); diacyl glyceride (DAG). The results are compiled from six independent, representative experiments. | | | | | | | | |

Recognition of mCD1d Transfected Tumor Cell Lines.

The results of our analyses using the mCD1d fusion protein suggested mCD1d-restricted T cells may require presentation of specific antigens for recognition of mCD1d molecules. Previous studies have demonstrated differences in the abilities of CD1d-restricted T cells to recognize different APCs, indicating that different APCs may present distinct antigens, and CD1d-restricted T cell clones may have heterogeneous antigen specificities (Brossay, L. et al, J. Immunol., 160:3681-8 (1998); Chiu, Y. H. et al., J. Exp. Med., 189:103-10 (1999); Couedel, C. et al., Eur. J. Immunol., 28:4391-7 (1998); Park, S. H. et al., J. Immunol., 160:3128-34 (1998)). Therefore, to investigate whether the antigen specificities of the hybridomas in the mCD1d fusion protein plate stimulation assay correlate with their ability to recognize mCD1d expressed by cells, we next tested the panel of hybridomas for recognition of four different mCD1d transfected tumor cell lines: RMA-S and EL-4 are derived from T lymphomas, A20 from a B lymphoma, and P815 from a mastocytoma. The hybridomas were incubated with the mCD1d transfected tumor cell lines, or the untransfected parental lines, without addition of exogenous antigens. The untransfected tumor cells stimulated little or no detectable IL-2 release by any of the hybridomas, whereas the mCD1d transfected cells could induce high levels of IL-2 secretion by certain NKT and diverse TCR hybridomas (Table 3).

TABLE 3
mCD1-Restricted Hybridoma Responses to mCD1d-Transfected Tumor Cells

| | Invariant TCRα NKT Hybridomas | | | | | | | | |
|---|---|---|---|---|---|---|---|---|---|
| | 24.8 A | 24 7.C | 24.9 E | DN32D3 | KT/7 | KT/12 | KT/22 | KT/23 | Vβ/9 |
| CD1/P815 | +++ | +++ | 0 | 0 | 0 | 0 | 0 | 0 | 0 |
| CD1/EL4 | +++ | +++ | +++ | + | 0 | 0 | 0 | + | 0 |
| CD1/RMA-S | +++ | ++ | 0 | 0 | 0 | 0 | 0 | 0 | 0 |
| CD1/A20 | ++ | +++ | 0 | 0 | 0 | 0 | 0 | 0 | 0 |

| | Diverse TCR Hybridomas | | | | | | | |
|---|---|---|---|---|---|---|---|---|
| | 14S 6.A | 14S.7.N | 14S 10.C | 14S 15.A | VII68 | VIII24 | XV19 | XV104 |
| CD1/P815 | +++ | + | + | +++ | ++ | ++ | + | + |
| CD1/EL4 | +++ | + | 0 | +++ | ++ | + | 0 | + |
| CD1/RMA-S | ++ | + | ++ | ++ | + | + | + | 0 |
| CD1/A20 | ++ | 0 | 0 | +++ | + | + | 0 | 0 |
| IL-2 secretion by mCD1d-restricted hybridomas in response to mCD1d-transfected tumor cell lines. The untransfected parental cell lines induced little or no detectable IL-2 production by any of the hybridomas. A “0” indicates a mean of less than 50 pg/ml IL-2 was secreted, “+” indicates 50-250 pg/ml, “++” | | | | | | | | |
| #indicates 250-1000 pg/ml, “+++” indicates greater than 1000 pg/ml IL-2 secretion. The results are compiled from three independent, representative experiments. | | | | | | | | |

Surprisingly, despite their common specificity for α-GalCer treated mCD1d fusion protein, there were three distinct patterns of recognition of the mCD1d transfected cell lines among the eight α-GalCer reactive NKT lineage hybridomas (Table 3). The α-GalCer reactive 24.7.C hybridoma recognized all of the mCD1d-expressing cells well (>500 pg/ml IL-2 release for each transfectant), while the 24.9.E, DN32D3, and KT23 hybridomas only responded to the mCD1d transfected EL-4 cell line (Table 3). The remaining four α-GalCer reactive hybridomas, KT7, KT12, KT22, and Vβ/9, showed little or no recognition of any of the mCD1d transfected cells (Table 3). The 24.8.A hybridoma, which had specificity for phospholipids rather than α-GalCer, responded well to all of the transfected cell lines (Table 3). All of the diverse TCR hybridomas recognized at least two of the mCD1d transfectants (Table 3). Thus, although the diverse TCR hybridomas did not respond strongly to any of the antigens screened in the mCD1d fusion protein stimulation assay, they could recognize mCD1d molecules expressed by different cell types. Additionally, hybridomas which shared specificity for α-GalCer, differed in their recognition of mCD1d expressed by distinct APCs.

Because cell surface mCD1d molecules may be complexed with cellular lipids, it has been difficult to evaluate the role of potential endogenous antigens in T cell recognition of mCD1d. The observation that a recombinant β₂m-linked mCD1d-IgGFc₂ₐ fusion protein did not stimulate high levels of IL-2 production from mCD1d-restricted T cell hybridomas, allowed us to develop a system to analyze the contribution of lipid antigens to recognition of mCD1d molecules by T cells. Activation of the hybridomas using plate-bound mCD1d fusion protein was dramatically enhanced after pre-incubation with certain lipids or lipid-containing cellular extracts. The response could be blocked by an anti-mCD1d mAb, showing the mCD1d molecule was required for stimulation. Pre-incubation of a negative control protein with the same lipids did not induce detectable IL-2 production, indicating that the lipids did not have a non-specific stimulatory effect. Hence, although other mechanisms cannot be ruled out, together these results suggest binding of certain lipid antigens to the plate-bound mCD1d molecules permitted efficient recognition of the mCD1d fusion protein by hybridomas expressing cognate TCRs.

Several investigations have now demonstrated that many NKT cells can respond to CD1d-mediated presentation of the unusual acylphytosphingolipid, α-GalCer (Brossay, L. et al, J. Immunol, 160:3681-8 (1998); Burdin, N. et al., J. Immunol., 161:3271-81 (1998); Kawano, T. et al., Science, 278:1626-9 (1997); Spada, F. M. et al, J. Exp. Med., 188:1529-34 (1998)). Additionally, glycosylated forms of PI have been implicated as determinants recognized by murine CD1d-restricted NKT cells during protozoal and mycobacterial infections, and PI containing compounds have been shown biochemically to be associated with mCD1d molecules purified from transfected human T2 cells (Apostolou, I. et. al., PNAS, 96:7610 (1999); Joyce, S. et al., Science, 279:1541-4 (1998); Schofield, L. et al., Science, 283:225-9 (1999)). Thus, sphingolipid and phospholipid compounds can apparently bind CD1d and function as antigens for CD1d-restricted NKT cells, but whether these molecules represent self or foreign antigens, and whether the NKT cells that respond to α-GalCer are the same as those that see phospholipids, has been unclear.

Our finding that a lipid extract of RMA-S cells could reconstitute the recognition of plate-bound mCD1d molecules by an NKT cell hybridoma shows that self lipids can serve as antigens for NKT cells. Further separation of the lipids within the organic phase extract revealed specificity for fractions containing mainly polar glycolipids and phospholipids, suggesting the 24.8.A hybridoma could recognize phospholipid antigens. This possibility was supported by experiments showing that the 24.8.A hybridoma responded to certain purified and synthetic phospholipids, including PI, PE, and PG, while PA, PS, and PC did not reproducibly induce IL-2 production. Whether the failure of PA, PS, and PC to stimulate IL-2 release resulted from lack of recognition by the 24.8.A hybridoma, or was due to inefficient binding of these lipids to the fusion protein under the conditions of the plate stimulation assay, is unclear. However, the 24.8.A hybridoma also did not respond to α-GalCer, which stimulated other hybridomas when added to the mCD1d fusion protein, indicating that it can bind. Thus, the 24.8.A hybridoma had specificity for three of the purified phospholipid antigens tested, but not for α-GalCer.

Unlike other hybridomas tested, the 24.8.A hybridoma had a variable amount of reactivity to the fusion protein which had not been pre-treated with a lipid antigen. This response could be due to recognition of the mCD1d molecule itself, independent of a specific antigen. Alternatively, the reactivity could result from recognition of an antigen that remained bound to the fusion protein after purification, that derived from the cells used to produce the fusion protein, or from the culture medium. Hence, given their abundance in cells and in culture supernatants, one of the phospholipids shown here to stimulate the 24.8.A hybridoma could also be responsible for its variable reactivity to the untreated fusion protein.

Eight of the NKT cell hybridomas tested responded strongly to α-GalCer pre-incubated with the mCD1d fusion protein. Surprisingly, four of these α-GalCer reactive hybridomas also had detectable reactivity to purified phospholipid antigens, suggesting the cellular antigens they recognize maybe related lipids. The eight diverse TCR hybridomas tested did not respond reproducibly to α-GalCer, but three also showed some response to purified phospholipids. In all, responses to PI, PE, or PG were detected for eight of the seventeen hybridomas tested. Thus, phospholipids may represent a major class of self antigens recognized by CD1d-restricted T cells, and some of the T cells that recognize these antigens may also respond to α-GalCer, while others do not.

The ability of the 24.8.A hybridoma to respond to phospholipids but not α-GalCer is particularly interesting with regard to its TCR gene usage. This hybridoma possesses a cannonically rearranged Vα14/Jα281 TCRα chain which is identical to those of the α-GalCer reactive NKT cell hybridomas, implying that it is the TCRβ chain which is responsible for its distinct antigen specificity. Surprisingly, the 24.8.A hybridoma expresses TCR Vβ 8.2, a Vβ gene which is also used by most of the α-GalCer reactive NKT cell hybridomas we tested (Table 1). Thus, it is unlikely that the Vβ of 24.8.A prevents recognition of α-GalCer, and seems instead that residues of the CDR3 loop encoded by the D segment, Jβ, or by N-region addition may be critical in conferring its antigenic specificity. Hence, despite their invariant TCR α chains and limited TCR Vβ gene usage, the diverse TCR β VDJ junctional regions of CD1d-restricted NKT cells may result in multiple different antigenic specificities within this T cell subset.

The potential for heterogeneous antigen specificities may explain our surprising finding that NKT cell hybridomas that responded similarly to α-GalCer presentation by the plate-bound mCD1d fusion protein, varied in their patterns of recognition of a panel of four mCD1d transfected tumor cells. One α-GalCer reactive hybridoma recognized all of the transfectants well, while three of the hybridomas only responded to one of the transfectants, and the remaining four α-GalCer specific hybridomas did not recognize any of the transfectants. This result suggests the endogenous cellular antigen recognized by these hybridomas is not α-GalCer or a single analogue, since in that case recognition of the mCD1d transfected cells should correlate with the α-GalCer reactivity observed in the plate stimulation assay. Instead, based on the three patterns of reactivity with the mCD1d transfectants, there must be at least three different antigenic specificities among the 8 α-GalCer reactive NKT cell hybridomas tested. The α-GalCer antigen might stimulate many NKT cells because it possesses a common determinant of some diverse set of antigens, or it may function similarly to a super antigen, and activate a large fraction of CD1d-restricted NKT cells, regardless of their other antigenic specificities. A recent analysis by Kawano et al. identifies an amino acid motif in the CDR3 region of TCRβ chains of human CD1d-restricted NKT cells that responded to selection by α-GalCer, indicating that this antigen preferentially stimulates a subset of the CD1d-restricted T cells (Kawano, T. et al., Int. Immunol., 11:881-7 (1999)).

Based on their diverse TCR structures, non-NKT lineage mCD1d-restricted hybridomas are thought to see a heterogeneous group of antigens (Behar, S. M. et al., J. Immunol., 162:161-7 (1999); Cardell, S. et al., J. Exp. Med., 182:993-1004 (1995)). The diverse TCR mCD1d-restricted hybridomas tested in this analysis could recognize multiple mCD1d transfected cell lines, suggesting they recognize broadly distributed cellular antigens. In contrast to most of the NKT hybridomas, the diverse TCR hybridomas did not respond strongly to α-GalCer. While this result suggests the diverse TCR population sees a set of antigens that is distinct from those recognized by mCD1d-restricted NKT cells, some of the diverse TCR hybridomas reacted to the same purified phospholipids recognized by members of the NKT cell subset. Therefore, some of the diverse TCR mCD1d-restricted T cell population may recognize similar self antigens to those recognized by mCD1d-restricted NKT cells.

The observation that mCD1d-restricted T cells varied in their recognition of different mCD1d transfected tumor cells, suggests antigens presented by mCD1d molecules differ according to the cell type. Given the broad expression of murine CD1d on cells of hematopoietic origin, variation in antigen presentation among cells that express mCD1d could be a critical mechanism of regulating mCD1d-restricted T cells (Brossay, L. et al., J. Immunol., 159:1216-24 (1997); Mandal, M. et al., Mol. Immunol., 35:525-36 (1998)). Little is known about the factors which affect endogenous lipid antigen presentation by mCD1d molecules, although variations in antigen presentation could arise from differences among APCs in expression, trafficking, processing, or mCD1d loading of antigens.

Antigen recognition in our mCD1d fusion protein presentation assay could occur after pre-incubation at pH 7.2, but was significantly enhanced by pre-incubation at pH 4.0. Therefore, while acidic pH is not required, it may facilitate lipid binding to the fusion protein. This observation might help to explain apparently conflicting results regarding α-GalCer presentation by APCs. Burdin et al. found that α-GalCer could be presented in the absence of endosomal trafficking and acidification, while in the experiments of Kawano et al. and Spada et al. these elements of cellular antigen processing appeared necessary for α-GalCer presentation to NKT cells (Burdin, N. et al., J. Immunol., 161:3271-81 (1998); Kawano, T. et al., Science, 278:1626-9 (1997); Spada, F. M. et al, J. Exp. Med., 188:1529-34 (1998)). Our results suggest that α-GalCer binding to mCD1d at the cell surface at neutral pH is possible, but that binding may be favored in endocytic vesicles which have an acidic pH. In contrast, recognition of cell surface mCD1d by diverse TCR hybridomas did not appear to require endosomal localization (Chiu, Y. H. et al., J. Exp. Med., 189:103-10 (1999)). Thus, intracellular trafficking of CD1d molecules may play a critical role in determining the antigens presented by cells that express CD1d.

The in vitro mCD1d-specific antigen recognition system described here, should prove useful in the isolation and identification of endogenous cellular antigens recognized by CD1 restricted T cells. Analysis of biochemically fractionated cellular lipids for their ability to stimulate mCD1d-restricted hybridomas after addition to the mCD1d fusion protein, could provide a means of identifying physiological antigens presented by normal or neoplastic cells. Identification of the natural antigens recognized by mCD1d-restricted T cells will be critical to our future understanding of the role of these cells in disease processes such as autoimmunity and cancer.

Experimental Procedures

Hybridomas.

The CD1d-restricted T cell clones 24.7, 24.8, 24.9 (NKT cell) and 14S.6, 14S.7, 14S.10, and 14S.15 (diverse TCRs) were all derived from spleen of wild type C57BL/6 mice, as described previously (Behar, S. M. et al., J. Immunol., 162:161-7 (1999)). To generate T cell hybridomas, the activated T cells were fused to the aminopterin-sensitive BW5147 αβTCR⁻ thymoma cell line using PEG1500, and hybrids were selected in HAT medium (Life Technologies, Gaithersburg, Md.). Resulting TT hybridomas were tested for recognition of RMA-S cells transfected with mCD1D1 compared to untransfected RMA-S cells, as described below. Hybridomas which demonstrated specific recognition of mCD1d were further subcloned by limiting dilution. The hybridomas are distinguished from the original T cell clones by the addition of a letter to their names. The KT/7, KT/12, KT/22, and KT/23 and Vβ/9 NKT cell hybridomas were derived from NK1.1⁺ T cells enriched from spleen of C57BL/6 mice by depletion of CD8+ T cells, naive T cells, and B cells by mAbs (anti-B220, CD8, and CD62L, or anti-CD8α, CD8β, and Me114) bound to magnetic microbeads, or to plastic. The purified cells were stimulated either by the anti-CD3 KT3 mAb (KT/7, KT/12, KT/22, KT/23), or by an anti-Vβ8.2 mAb (Vβ/9), and addition of IL-2 or IL-2 and IL-7. After 4-5 days of culture the cells were fused with BW5147 thymoma cells. The VII68, VIII24, XVI9, and XV104 diverse TCR hybridomas were generated from CD4⁺ T cells from class 110 mice, as described previously (Cardell, S. et al., J. Exp. Med., 182:993-1004 (1995)). The DN32D3 hybridoma was derived as described (Lantz, O., and Bendelac, A., J. Exp. Med., 180:1097-106 (1994).

Generation of mCD1d Fusion Protein.

A soluble murine CD1d fusion protein covalently linked to human β2m at the N-terminus by a glycine-serine (gly-ser) spacer peptide, and at the C-terminus to the Fc portion of murine IgG₂ₐ by another gly-ser spacer peptide, was constructed as follows. All synthetic oligonucleotides were commercially obtained, (Operon Technologies, Emeryville, Calif.). A cDNA of the full length coding sequence of mCD1D1 was used as template DNA for PCR amplification. PCR primers were designed to create a truncated mCD1D1 gene which eliminates the cytoplasmic, transmembrane, and leader peptide sequences. The 5′ primer oligonucleotide sequence, containing a Spe I restriction site, was 5′-GCGCGGACTAGTTCTGAAGCCCAGCAAAAGAATTACACC-3′ (Seq. ID. No.6), and the 3′ primer sequence, containing a Not I restriction site, was5′-TGCTTGGCGGCCGCTCCAGTAGAGGATGATATCCTGTCC-3′ (Seq. ID. No.7). A cDNA fragment encoding human β₂m fused to the gly-ser linker was generated by PCR, using as a template a cDNA construct encoding a human β₂m-linked single chain CD1a molecule. The 5′ primer sequence containing an Xho I site was: 5′-GCGCGGCTCGAGCATGTCTCGCTCCGTGGCCTTAGC-3′ (Seq. ID. No. 8), and the 3′ primer sequence containing an Xba I restriction site was: 5′-CGGCTCTAGATCCACCTCCAGAACCGGATCCACCTG-3′ (Seq. ID. No. 9). The PCR products were digested with the appropriate restriction enzymes, ligated and subcloned, and the fragment containing β₂m linked to mCD1d was excised by digestion with Xho I and Not I. This fragment was linked to a cDNA fragment encoding the hinge, CH₂, and CH₃ regions of murine IgG₂ₐ using a synthesized DNA fragment encoding a 14 amino acid gly-ser spacer peptide sequence (SGPGGSGGSGGSGG) (Seq. ID No. 10), made from the following complementary oligonucleotides: 5′-GGCCCGGGAGGTTCTGGAGGTTCAGGAGGTTCTGGAGGG-3′ (Seq. ID. No. 11), and 5′-GATCCCCTCCAGAACCTCCTGAACCTCCAGAACCTCCCG-3′ (Seq. ID. No. 12). The 3 cDNA fragments were ligated and subcloned into the pBluescript SK vector (Stratagene, La Jolla, Calif.). The resulting construct was fully sequenced with M13 reverse and T7 outside primers, to ensure that no coding mutations were present, then excised by restriction digestion and subcloned into the pBJ1-neo expression vector for transfection (Lin et al., 1990).

Production and Purification of mCD1d Fusion Protein.

Chinese Hamster Ovary (CHO) cells were transfected with the PBJ1-neo vector containing the β₂m-mCD1d-Fc₂ₐ cDNA construct by electroporation, then selected for G418 drug resistance and subcloned by limiting dilution to isolate stably transfected cells with high protein expression levels. Culture supernatants were tested for the presence of the mCD1d fusion protein by a standard double antibody sandwich ELISA using the 1B1 anti-mCD1d monoclonal antibody (Pharmingen, San Diego, Calif.) as a capture reagent, and a biotinylated polyclonal rabbit anti-human β₂m anti-serum (DAKO, Glostrup, Denmark) followed by a streptavidin-alkaline phosphatase conjugate (Zymed, South San Francisco, Calif.), or an anti-murine IgG₂ₐ antibody conjugated directly to alkaline phosphatase (Zymed), as the detection reagent. The fusion protein was detectable by both methods, indicating the mCD1d was complexed with both human β₂m and murine IgG₂ₐ Fc. The CD1d fusion protein was purified by passage over a protein A sepharose column (Amersham-Pharmacia Biotech, Piscataway, N.J.), and eluted with 50 mM Sodium Acetate buffer at pH 4.3, followed by immediate neutralization by addition of 1/10 volume of a 1M Tris buffer at pH 8.8. Subsequent analysis of the protein A eluate by size exclusion chromatography using a Superose 6 column (Amersham-Pharmacia) revealed a single peak eluting slightly earlier than a polyclonal IgG standard, as expected for a homodimeric fusion protein complex. Analysis by reducing and non-reducing SDS-PAGE demonstrated single bands at the expected molecular weights of approximately 100 kD and 200 kD, respectively.

Cellular Extracts and Fractionation.

Cellular lipid was extracted from RMA-S and S49 murine T lymphoma cells using the method of Folch et al., with modifications as described by Hamilton and Hamilton (Folch, J. et al., J. Biol. Chem., 226:497-509 (1956); Hamilton, S. et al., Oxford: IRL Press at Oxford University (1992)). Briefly, 1 g of pelleted cells was mixed with 20 ml of a 2:1 v/v chloroform: methanol solution (C:M), then homogenized and incubated at RT for one hour. The mixture was centrifuged to remove insoluble material, and the supernatant saved. A ⅕ volume of sterile dH₂O was added to the C:M supernatant and the mixture was shaken until an emulsion formed, then incubated 24 hr at RT to allow phase separation into an organic fraction, an aqueous fraction, and the interface. For analysis using the mCD1d fusion protein assay, the aqueous and interface fractions were lyophilized, and the organic fraction was dried under a stream of nitrogen. The samples were then quantified by weight and resuspended in DMSO. The organic phase was further fractionated by dissolving 35 mg of dried sample in chloroform and applying it to a silica column (400 mesh silicic acid, Selecto Scientific, Ga.). Lipids of increasing polarity were eluted from the column using a stepwise gradient of chloroform, acetone, and methanol. The resulting fractions were dried, quantitated, and solubilized in C:M, then dried down and resuspended in DMSO prior to use.

Glycolipid Antigens.

The following antigens were commercially obtained (Matreya Corporation, Pleasant Gap, Pa.): purified bovine brain sphingomyelin (Sph), purified bovine brain disialoganglioside (GD1a), purified bovine brain gangliotriosyl ceramide (aGM2), purified plant monogalactosyl diglyceride (MGDG), purified bovine phosphatidylserine (PS), purified soybean phosphatidylinositol (PI), synthetic dipalmitoylphosphatidylinositol 3-phosphate (PI3-P), synthetic dipalmitoylphosphatidylinositol bis-3,4-phosphate (PI3,4-P2), synthetic dipalmitoyl phosphatidylinositol tris-3,4,5-phosphate (PI3, 4, 5-P3), synthetic distearoyl phosphatidylcholine (PC), purified distearoylphosphatidylethanolamine (PE), synthetic dipalmitoyl phosphatidylglycerol(PG), and synthetic dipalmitoyl phosphatidic acid (PA). Palmitic acid (palmitate), free inositol, and dipalmitindiacylglycerol (DAG) were acquired from Sigma (St Louis, Mo.). The synthetic α and β-galactosylceramide (α-GalCer, β-GalCer), and unglycosylated ceramide (Cer) were produced synthetically as previously described (Kawano et al., 1997). The antigens were dissolved at a stock concentration of 100 or 200 μg/ml in DMSO and were sonicated in a 37° C. water bath for 10 minutes prior to use.

Plate-bound mCD1d Fusion Protein Hybridoma Stimulation Assays

To test for recognition of the mCD1d fusion protein and purified or synthetic antigens, 96 well protein A coated plates (Pierce Chemical Company) were incubated with 400-600 ng/well of the fusion protein or a negative control IgG₂ₐ antibody, RPC5.4 or UPC10, in PBS, at pH 7.2. Lipid antigens were diluted into PBS and added where specified at the indicated molar ratio of antigen to fusion protein, (when not specified the ratio was 40:1). Protein A plates containing the fusion protein and antigen were incubated 24-48 hr at 37° C., then washed three times with 200 μl/well sterile PBS, pH 7.2, and two times with 200 μl/well sterile culture medium (containing RPMI supplemented with L-glutamine and penicillin/streptomycin, Life Technologies, Gaithersburg, Md., and 10% bovine calf serum, Hyclone Laboratories, Logan, Utah). For assays in which the PI was phospholipase treated, it was first diluted into 0.01M Tris, 0.15M NaCl, pH 7.5, containing 0.25 U PI-specific phospholipase C or 0.5 U phospholipase D (Sigma, St Louis, Mo.), and incubated 30 minutes at room temperature, then added to the protein A plates as described above. For assays in which the pH was varied during antigen incubation with the fusion protein, the fusion protein and α-GalCer were diluted into a 20 mM citrate/phosphate buffer of the specified pH, which contained 0.15 M NaCl, and after incubation, the samples were neutralized by addition of 1M Tris, pH 7.5. Hybridoma cells were added to fusion protein/antigen treated plates at a concentration of 1×10⁵ cells/well, in a total volume of 150 μl/well. Assays were performed using 2-6 replicate wells. In some assays, an anti-mCD1d blocking antibody (19G11) was included at a final concentration of 20 μg/ml. The plates were incubated at 37° C. for 16-20 hr, and culture supernatants were withdrawn for analysis. Each experiment was performed at least three times.

Generation of mCD1d APC Transfectants and mCD1d Recognition Assay.

CD1D1 transfected RMA-S cells were derived as described previously (Behar, S. M. et al., J. Immunol., 162:161-7 (1999)). A similar procedure was used to transfect the EL4, A20, and P815 cell lines. Briefly, the cells were transfected by electroporation with the pSRα-neo expression vector containing mCD1D1 cDNA, and subjected to G418 drugs election, to obtain stably transfected lines. Drug resistant cells were stained using the 19G11 or 1B1 rat anti-mCD1d mAbs (Dr. Albert Bendelac, Princeton University, and Dr. Laurent Brossay, UCLA, respectively), and analysed by flow cytometry. In some cases the cultures were sorted using a FAC sort (Becton Dickinson, Raritan, N.J.) to obtain cells expressing high levels of mCD1d, then cloned by limiting dilution. Hybridomas were tested for IL-2 production in the presence of the mCD1d transfected compared to the untransfected parental cell lines. Hybridomas and APCs were added at a concentration of 1×10⁵ cells/well each, in a total volume of 150 μl/well, and incubated as described above.

Detection of IL-2 Secretion.

IL-2 secreted in the hybridoma stimulation assays was quantitated in a double antibody sandwich ELISA, by comparison to a standard curve of purified murine IL-2 (Pharmingen, San Diego, Calif.). Hybridoma plate stimulation supernatants (used either neat or diluted) and serially diluted IL-2 standards were added to 96 well ELISA plates coated with a rat anti-mouse IL-2 capture antibody (Pharmingen). IL-2 was detected by addition of a biotinylated rat anti-mouse IL-2 antibody, followed by addition of a streptavidin-alkaline phophatase conjugate, and a chromogenic substrate. The pg/ml of IL-2 present in the hybridoma supernatants was quantitated by linear regression of the IL-2 standard curve.

### Example 2

### Multivalent Soluble CD1 Fusion Protein

One aspect of the invention is a stably folded soluble CD1 fusion protein that is multivalent and can be fluorescently labeled, and which can be loaded with lipid or glycolipid antigens in vitro and used to stain or functionally investigate cognate T cells. Such fusion proteins of human CD1d, and murine CD1d have been created and tested. To make the fusion proteins, new cDNA constructs were generated that encode human β₂m attached by a glycine-serine spacer peptide to the N-terminus of the extracellular domains of CD1. The C-terminus of the CD1 molecule is fused by another glycine-serine spacer peptide to the hinge and CH2—CH3 domains of murine IgG₂ₐ. The cDNA constructs were cloned into the pBJ1-neo expression vector, for stable expression in mammalian cells. (Lin, A. et al., Science, 249:677-679 (1990)). The fusion proteins are expressed in CHO cells, and purified from the culture supernatant using a protein A affinity column and pH 4.3 acid buffer elution. Analysis by SDS-PAGE and size exclusion chromatography indicate the fusion proteins are secreted as glycosylated, disulfide-linked dimers of the expected molecular weight of approximately 200 kM. Using a standard double antibody sandwich ELISA technique, the fusion proteins can be detected with mAb specific for native CD1d molecules, human β2m, and murine IgG₂ₐ.

The fusion proteins can be coated on plastic and used to investigate the functional reactivity of CD1-restricted T cells to specific lipid antigens, as shown in Example 1.

To facilitate binding to CD1 specific T cells for detection by flow cytometry, a highly multimerized form of the CD1d fusion protein is formed using fluorescently labeled protein A molecules. Protein A molecules spontaneously associate in solution at neutral pH with immunoglobulin Fc regions, forming complexes containing four Fc molecules and two protein A molecules (4+2 complexes, Langone, J.J. et al, Molec. and Cell. Biochem, 65(2):159-70 (1985)). The human CD1d-Fc fusion protein was incubated with Alexa 488-dye labeled protein A, and the 4+2 complexes purified by size exclusion chromatography on a Phannacia Superose 6 column using PBS pH 7.2 as a running buffer. The purified 4+2 aggregates are concentrated to 100 μg/ml with ovalbumin as a carrier protein. The CD1d-Fc aggregate is then pre-incubated for 24 to 48 hours at 37° C. with antigenic glycolipids dissolved in DMSO at a 40:1 molar ratio of lipid to fusion protein, or with an equivalent volume of DMSO alone as a negative control. The T cell staining is performed at room temperature or 4° C. for 20 min, at a concentration of 40 μg/ml of the lipid or control treated CD1d-Fc aggregate.

### Example 3

### Screening/Diagnostic Assay

To test the specificity of staining, previously isolated human CD1d-restricted T cell clones (Porcelli, S. et al., Nature, 341(6241):447-50 (1989)) were stained with CD1d-Fc aggregates treated with lipid antigens or control compounds. Flow cytometric analysis showed that the CD1d fusion protein aggregates treated with specific lipid antigens such as α-galactosyl ceramide (α-GalCer), and α-glucosyl ceramide (α-GIcCer) gave positive staining, whereas the CD1d-Fc aggregates treated with the related lipids α-mannosyl ceramide (α-ManCer), α-galactosyl ceramide (α-GalCer), ceramide (Cer), or DMSO alone did not stain above background levels. This experiment demonstrates the requirement for treatment of the CD1d fusion protein with specific lipid antigens to enable stable binding to cognate T cells. Furthermore, the lipid antigen specificity in these staining experiments correlates precisely with the functional reactivity to lipid antigens presented by CD1d molecules previously observed for these T cell clones (Kawano, T. et al., Science, 278(5343):1626-9 (1997); Spada, F. M. et al., J. Exp. Med., 188(8):1529-34.1 (1998)). The specificity of staining was further confirmed by comparing staining of 2 CD1d-restricted T cell clones with that of 4 T cell clones that are not CD1d-restricted. The lipid antigen treated fusion protein positively stains the CD1d-restricted T cells, but did not stain the non-CD1d-restricted T cells above background levels.

Flow cytometric analysis of a CD1d-restricted T cell clone stained with the multimerized CD1d-Fc fusion protein (abbreviated as “hd(8)-fl”) was performed as follows. Staining with CD1d-Fc treated with lipid antigens dissolved in DMSO was compared with CD1d-Fc treated with DMSO alone as a negative control. The specific lipid antigen used were: aGalCer is α-galactosyl ceramide (KRN7000); aGlcCer is α-glucosyl ceramide; aManCer is α-mannosyl ceramide; bGalCer is β-galactosyl ceramide; Cer is ceramide (acylphytosphingolipid). Note that positive staining of the CD1d-restricted T cell clone is only observed when the CD1d-Fc fusion protein is treated with aGalCer, but not with the other related lipids, or with DMSO alone.

Flow cytometric analysis of a series of human T cell clones stained with the multimerized CD1d-Fc fusion protein, treated with α-GalCer or DMSO alone also was performed, including staining of two different CD1d-restricted (“NKT”) T cell clones DN2.B9 and DN1.10B3. Four other T cell clones that are not CD1d-restricted also were stained. Positive staining with the α-GalCer treated CD1d fusion protein was seen for the two CD1d-restricted clones, but no staining is seen for the other 4 non-CD1d-restricted T cell clones.

To investigate whether the lipid loaded fusion protein can detect CD1d reactive T cells in peripheral blood, three color flow cytometric analysis was performed on PBMCs purified from a healthy donor. The cells were stained with anti-CD3, anti-CD161, and the α-GalCer antigen loaded or DMSO treated CD1d-Fc aggregates, or an aggregate made with a negative control antibody (UPC10). The CD1d-Fc aggregate treated with α-GalCer stained about 6-fold as many T cells as the CD1d-Fc treated with DMSO alone, and about 10-fold as many as the UPC10 negative control. A population of CD3⁻ lymphocytes was stained by all three protein A aggregated reagents, suggesting this was due to non-specific binding. However, very few CD3+ cells were stained by the negative control UPC10 complex, indicating very low non-specific binding of this type of staining reagent to T cells. This experiment suggests that this reagent can be used to detect lipid antigen specific CD1d-restricted T cells directly in peripheral blood samples.

T cell lines and clones stained with the α-GalCer treated CD1d-Fc aggregates were isolated from peripheral blood by flow cytometric cell sorting and limiting dilution cloning, and cultured using standard techniques. Functional analysis of the T cell lines and clones revealed that they secrete cytokines in response to CD1d-transfected antigen presenting cells, but not to the untransfected parent cells. Cytokine secretion was enhanced in the presence of α-GalCer. This experiment shows that T cells isolated using the α-GalCer treated CD1d-Fc fusion protein are CD1d-restricted, and can recognize CD1d molecules at the cell surface of antigen presenting cells that may be complexed with endogenous lipid antigens, and that the T cells also respond strongly to the α-GalCer lipid antigen.

Three color flow cytometric analysis of peripheral blood lymphocytes from a healthy donor was performed with the X-axes showing anti-CD3 staining, the Y-axes show staining with: the UPC10 negative control complex; the CD1d-Fc complex treated with DMSO; the CD1d-Fc complex treated with α-GalCer. The percentage of the total lymphocytes contained within the quadrant was obtained. There was an increased number of cells stained using α-GalCer treated CD1d-Fc complex compared to CD1 Fc treated with DMSO alone, or the negative control antibody complex.

### Example 4

### Diagnostic Methods

a.) Enumeration of Antigen specific CD1-restricted T Cells for Evaluation of Autoimmune Disease Progression.

The fluorescent CD1 fusion protein is treated with α-GalCer lipid antigen (or other CD1 antigen that is an endogenous mammalian autoantigen) and used with anti-CD3 antibodies, and/or other T cell antigen antibodies, to stain purified peripheral blood mononuclear cells for multicolor flow cyometric analysis (as described above). The number of cells stained positively with the CD1 fusion protein aggregate is compared to standard values obtained for normal individuals.

b.) Investigation of the Functional Phenotype of Antigen Specific CD1-restricted T Cells for Evaluation of Autoimmune Disease Progression.

In papers such as Wilson, S. B. et al., Nature, 391(6663):177-81 (1988), it has been shown that CD1-restricted T cells of individuals who have progressed to autoimmune diabetes differ from those of non-progressers in that they have a strong THI bias. Therefore the ability to test the TH1/TH2 polarization of CD1-restricted T cells is believed to be an important diagnostic tool in evaluating autoimmune disease progression. To do this, purified peripheral blood lymphocytes are stimulated to produce cytokines by, for example, phorbol esters plus a calcium ionophore, or by phytohemaglutinin (as described in Pharmingen product literature). The cells are then stained with the lipid antigen (α-GalCer) treated fluorescent CD1 fusion protein aggregate and an anti-CD3 antibody, and then fixed and permeabilized and stained with antibodies for cytokines of interest such as γ-interferon and IL-4. (The intracellular cytokine staining can be accomplished with a kit available form Pharmagen). This allows determination of the TH1/TH2 cytokine polarization of the population of CD1-restricted antigen-specific T cells compared to the rest of the T cells.

Alternatively, three color staining can be performed using the lipid antigen treated CD1 fusion protein, anti-CD3, and anti-chemokine receptor antibodies that have been shown to correlate with TH1 or TH2 cytokine polarizatin (CCR5 and CCR3 respectively, (Lanzavecchia and Sallusto, Curr. Opin. Immunol., 12(1):92-8 (2000)).

### Example 5

### Therapeutic Methods:

a.) Activation of Antigen Specific CD1-restricted T Cells for Immunotherapeutic Treatment of Disease (Autoimmune Disease, Cancer, Allergy, Viral Infections, Bacterial Infections).

CD1-restricted antigen-specific T cells are selected by staining with the CD1 antigen treated CD1 fusion protein aggregate and CD3 as described above, and sterilely sorted by flow cytometry. The sorted T cells are cultured with standard tissue culture medium containing phytohemagglutinin (PHA), IL-2, and irradiated autologous or allogeneic purified peripheral blood mononuclear “feeder” cells. This method causes the sorted T cells to proliferate in culture and therefore results in the expansion (and activation) of antigen-specific CD1-restricted T cells that can then be administered to patients for immunotherapy.

b.) Depletion of Antigen Specific CD1-restricted T Cells for Immunotherapeutic Treatment of Disease (Autoimmune Disease, Cancer, Allergy, Viral Infections, Bacterial Infections).

In this application the cell stained by the CD1 lipid antigen treated CD1 fusion protein aggregate are sorted out from the rest of the T cells and discarded, and the remaining T cells are readministered to the patient. Alternatively, a toxin is attached to the CD1 fusion protein and the antigen treated fusion protein aggregate is administered in vivo, to kill antigen specific CD1-restricted T cells.

### Equivalents

It should be understood that the preceding is merely a detailed description of certain embodiments. It therefore should be apparent to those of ordinary skill in the art that various modifications and equivalents can be made without departing from the spirit and scope of the invention, and with no more than routine experimentation. It is intended to encompass all such modifications and equivalents within the scope of the appended claims.

All references, patents and patent applications that are recited in this application, including priority documents, are incorporated by reference herein in their entirety.

We claim:

1. A method for identifying an antigen recognized by a CD1-restricted T cell, comprising:
(a) contacting a CD1 fusion protein with a putative CD1 antigen under conditions to form a CD1-presented antigen complex;
(b) contacting the CD1-presented antigen complex with a CD1-restricted T cell under conditions to allow complex-mediated activation of the T cell; and
(c) detecting activation of the T cell, wherein activation indicates that the putative CD1 antigen is recognized by the CD1 restricted T cell.

2. The method of claim 1, wherein the CD1 fusion protein is selected from the group consisting of a CD1a fusion protein, a CD1b fusion protein, a CD1 c fusion protein, and a CD1d fusion protein.

3. The method of claim 1, wherein the CD1 fusion protein is a CD1d fusion protein.

4. The method of claim 1, wherein at least one contacting step (a) or (b) is performed in vitro.

5. The method of claim 1, wherein at least one contacting step (a) or (b) is performed in vivo.

6. The method of claim 1, wherein the CD1 fusion protein is multimeric.

7. The method of claim 1, wherein the CD1 fusion protein is bound to protein A.

8. The method of claim 1, wherein the CD1 fusion protein is immobilized.

9. The method of claim 1, wherein the CD1 fusion protein is soluble.

10. The method of claim 1, wherein the CD1 fusion protein is soluble and contains a detectable label.

11. The method of claim 1, wherein the putative CD1 antigen is a naturally-occurring, lipid-containing molecule.

12. The method of claim 1, wherein the putative CD1 antigen is a synthetic molecule.

13. The method of claim 1, wherein the putative CD1 antigen is contained in or isolated from a sample selected from the group consisting of: a mammalian cell, a plant cell, a bacteria, a virus, a fungus, a protist, and a synthetic library.

14. The method of claim 1, wherein the putative CD1 antigen is contained in or isolated from a total lipid extract of a sample selected from the group consisting of: a mammalian cell, a plant cell, a bacteria, a virus, a fungus, a protist, and a synthetic library.

15. The method of claim 1, wherein the putative CD1 antigen is contained in or derived from a mammalian cell.

16. The method of claim 15, wherein the mammalian cell is contained in or derived from a sample selected from the group consisting of: a blood sample, a cerebrospinal fluid sample, a synovial fluid sample, a tissue sample, a urine sample, an amniotic fluid sample, a peritoneal fluid sample, and a gastric fluid sample.

17. The method of claim 1, wherein the putative CD1 antigen is a lipid-containing molecule selected from the group consisting of: a polar lipid (e.g., a ganglioside, a phospholipid); a neutral lipid, a glycolipid; and a lipidated protein or lipidated peptide.

18. The method of claim 1, further comprising the step of removing the putative CD1 antigen that is not present in the CD1-presented antigen complex.

19. The method of claim 1, wherein the CD1-restricted T cell is selected from the group consisting of (a) a mouse CD1-restricted T cell; and (b) a human CD1-restricted T cell.

20. The method of claim 1, wherein the CD1-restricted T cell is a mouse NKT cell.

21. The method of claim 1, wherein the CD1-restricted T cell is selected from the group consisting of: DN1.10B3; DN2.B9; DN2.D5; and DN2.D6.

22. The method of claim 1, wherein detecting activation of the T cell comprises detecting one or more of an indicator selected from the group consisting of: (a) binding of the CD1-restricted T cell to the complex; (b) a change in cytokine release by the CD1-restricted T cell; (c) a change in calcium flux in the CD1-restricted T cell; (d) a change in protein tyrosine phosphorylation flux in the CD1-restricted T cell (e) phosphatidyl inositol turnover flux in the CD1-restricted T cell.

23. The method of claim 1, wherein detecting activation of the T cell comprises detecting binding of the T cell to the complex.

24. The method of claim 1, wherein the CD1 fusion protein is soluble and contains a detectable label and wherein detecting activation of the T cell comprises detecting binding of the CD1-restricted labeled T cell to the labeled CD1 fusion protein.

25. The method of claim 1, wherein detecting activation of the T cell comprises detecting cytokine release by the T cell.

26. The method of claim 1, wherein detecting cytokine release comprises detecting release of one or more cytokines selected from the group consisting of: an interferon (e.g., IFN-gamma); an interleukin (e.g., IL-2, IL-4, IL-10, IL-13); a tumor necrosis factor (e.g., TNF-alpha); and a chemokine.

27. The method of claim 1, further comprising the step of contacting the T cell with a co-stimulatory agent prior to detecting activation of the T cell.

28. The method of claim 15, wherein the co-stimulatory agent selected from the group consisting of: (a) an adhesion molecule (e.g., CD2); (b) an NK complex molecule (e.g., CD161, CD94); (c) an antibody to the T cell receptor (e.g., an anti-CD3 antibody); (d) a non-specific stimulator (e.g., phytohemaglutinin (“PHA”), concanavalin A (Con A”); phorbol myristate acetate (“PMA”); (e) an antigen-presenting cell which does not express CD1; and (f) a co-stimulatory molecule (e.g., CD28).

29. A method for identifying a CD1-restricted T cell, comprising:
(a) contacting a CD1-presented antigen complex with a putative CD1-restricted T cell under conditions to allow complex mediated activation of the putative CD1-restricted T cell; and
(b) detecting activation of the putative CD1-restricted T cell, wherein activation indicates that the putative CD1-restricted T cell is a CD1-restricted T cell.

30. The method of claim 29, wherein the CD1-presented complex contains a detectable label.

31. The method of claim 30, wherein detecting activation of the putative CD1-restricted T cell comprises detecting binding of the CD1-restricted T cell to the labeled CD1 fusion protein.

32. The method of claim 31, wherein detecting comprises detecting the labeled T cells bound to the labeled CD1 fusion protein by flow cytometry.

33. The method of claim 29, wherein the putative CD1-restricted T cell is contained in a biological sample.

34. The method of claim 33, wherein the biological sample is selected from the group consisting of a blood sample, a cerebrospinal fluid sample, a synovial fluid sample, a tissue sample, a urine sample, an amniotic fluid sample, a peritoneal fluid sample, and a gastric fluid sample.

35. A method for detecting a CD1-restricted T cell activity in a sample, comprising:
(a) contacting a CD1-presented antigen complex with a sample suspected of contacting a CD1-restricted T cell under conditions to allow complex mediated activation of the CD1-restricted T cell; and
(b) detecting a CD1-restricted T cell activity;
wherein the CD1-restricted T cell activity is selected from the group consisting of: (1) the number of CD1-restricted T cells as a percentage of the total T cell population or a change in said number; and (2) a CD1-restricted T cell functional activity or a change in said functional activity.

36. The method of claim 35, wherein detecting a CD1 restricted T cell activity comprises detecting the number of CD1 restricted T cells or a change in said number.

37. The method of claim 36, wherein the CD1-presented complex contains a detectable label.

38. The method of claim 37, wherein detecting the number of CD1 restricted T cells comprises detecting the CD1-presented complex containing a detectable label bound to the CD1-restricted T cell.

39. The method of claim 38, wherein detecting comprises detecting the labeled T cell by flow cytometry.

40. The method of claim 35, wherein detecting a CD1 restricted T cell activity comprises detecting a CD1 restricted T cell functional activity or a change in said functional activity.

41. The method of claim 35, wherein the CD1-restricted functional activity is selected from the group consisting of: (a) binding of the CD1 restricted T cell to the complex; (b) cytokine release by the CD1 restricted T cell; (c) calcium flux in the CD1 restricted T cell; (d) protein tyrosine phosphorylation in the CD1 restricted T cell; (e) phosphatidyl inositol turnover in the CD1 restricted T cell.

42. The method of claim 35, wherein the sample is selected from the group consisting of a blood sample, a cerebrospinal fluid sample, a synovial fluid sample, a tissue sample, a urine sample, an amniotic fluid sample, a peritoneal fluid sample, and a gastric fluid sample.

43. A composition comprising
a vaccine comprising an immunogen that: (1) binds to a CD1 molecule, and (2) enhances or induces protective immunity to a condition,
a CD1 fusion protein that selectively binds to the immunogen to form a CD1-presented immunogen complex that activates a cognate CD1-restricted T cell; wherein the CD1 fusion protein is present in an amount effective to enhance or induce protective immunity to the condition, and
a pharmaceutically acceptable carrier.

44. The composition of claim 43, wherein the CD1 fusion protein is multivalent.

45. The composition of claim 43, wherein the condition is an infectious disease.

46. The composition of claim 43, wherein the condition is an infectious disease and the immunogen is derived from an infectious agent selected from the group consisting of a bacterial infectious agent, a viral infectious agent, a fungal infectious agent, and a protist infectious agent.

47. The composition of claim 43, wherein the condition is a cancer.

48. The composition of claim 43, wherein the condition is a cancer and the immunogen is derived from a cancer cell.

49. The composition of claim 43, wherein the condition is an autoimmune disease.

50. The composition of claim 43, wherein the condition is an autoimmune disease and the immunogen is derived from a selective marker for the autoimmune disease.

51. The composition of claim 43, wherein the disorder is an allergy.

52. The composition of claim 43, wherein the disorder is an allergy and the immunogen is derived from an allergen.

53. A method for treating a condition, comprising:
(a) administering the composition of claim 43 to a subject in need of such treatment in an amount effective to treat the condition.

54. A method for enhancing vaccine-induced acquired protective immunity, comprising
administering to a subject a CD1 fusion protein in combination with a vaccine that enhances or induces protective immunity to a condition.

55. The method of claim 54, wherein the CD1 fusion protein is administered subsequent to administering the vaccine to enhance recall of protective immunity.

56. The method of claim 54, wherein the vaccine enhances or induces protective immunity to a microbial infectious disease.

57. The method of claim 56, wherein the vaccine enhances or induces protective immunity to a tumor antigen, an allergen, or an autoantigen.

58. The method of claim 54, wherein the condition is selected from the group consisting of: an infectious disease, an allergic response, an autoimmune disorder, and a cancer.

59. A method of activation of antigen specific CD1-restricted T cells for immunotherapeutic treatment of disease, comprising:
(1) selecting antigen specific CD1-restricted T cells; and
(2) sterilely sorting the selected CD1-restricted T cells by flow cytometry.

60. The method of claim 59, wherein selecting antigen specific CD1-restricted T cells comprises staining with the CD1-restricted T cell antigen complexes of the invention.

61. The method of claim 59, further comprising the step of costimulating with a stimulatory agent prior to sterilely sorting the selected CD1-restricted T cells.

62. The method of claim 59, further comprising the step of (3) expanding the selected T cells in culture.

63. The method of claim 62, further comprising the step of administing the expanded T cells to a subject in need of such treatment.

64. A method for depleting antigen specific CD1-restricted T cells for immunotherapeutic treatment of disease, comprising:
(1) selecting antigen specific CD1-restricted T cells; and
(2) sterilely sorting out (removing) the selected CD1-restricted T cells.

65. The method of claim 64, further comprising the step of (3) administering to a subject the T cells which are not antigen specific CD1-restricted T cells.

66. The method of claim 64, further comprising the step of: (3) attaching a toxin to the antigen specific CD1-restricted T cells; and (4) administering the toxin-labeled cells to the subject
